(19) United States
(12) Patent Application Publication
(10) Pub. No.: US 2024/0415655 A1
(43) Pub. Date: Dec. 19, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Kevin Gantz, Laguna Beach, CA (US); Eric Michael Oberwise, Newport Beach, CA (US)

(21) Appl. No.: 18/815,425

(22) Filed: Aug. 26, 2024

Related U.S. Application Data
(63) Continuation of application No. PCT/US2023/013973, filed on Feb. 27, 2023.
(60) Provisional application No. 63/315,958, filed on Mar. 2, 2022.

(51) Int. Cl. A61F 2/24; A61M 25/01

(52) U.S. Cl. CPC A61F 2/2466; A61F 2/246; A61F 2/2463; A61M 25/0105; A61F 2220/0008; A61F 2250/0004; A61M 2205/584

(57) ABSTRACT

A handle assembly for controlling the implantable device includes an actuation element extending through a sheath, a paddle actuation control coupled to the actuation element, and a visual indicator. In some implementations, the handle further includes a paddle width adjustment element and a paddle width control that is actuatable to cause axial movement of the paddle width adjustment element, thereby causing a width of the at least one of the pair of paddles of the implantable device to be moved from a first width to a second width. In some implementations, the visual indicator is coupled to the actuation element or the paddle width adjustment element through the handle housing and is configured to provide an indication regarding a configuration of the implantable device.

### RELATED APPLICATIONS

The present application is a continuation of Patent Cooperation Treaty application no. PCT/US2023/013973, filed on Feb. 27, 2023, which claims the benefit of U.S. Provisional Patent Application No. 63/315,958, filed on Mar. 2, 2022, titled “Heart Valve Repair Devices and Delivery Devices Therefor”, which is incorporated herein by reference in its entirety for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves may be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure may be included in the examples summarized here.

In some implementations, there is provided an implantable device or implant (e.g., implantable device, etc.) that is configured to be positioned within a native heart valve to allow the native heart valve to form a more effective seal.

In some implementations, an implantable device or implant includes an anchor portion. Each anchor includes a plurality of paddles that are each moveable between an open position and a closed position.

In some implementations, a handle assembly for controlling a transvascular implantable device includes a handle housing, a sheath extending distally from the handle housing, an actuation element extending through the sheath, a paddle actuation control (e.g., knob, button, switch, lever, etc.) coupled to the actuation element, and at least one visual indicator coupled to the actuation element through the handle housing.

In some implementations, the paddle actuation control is configured such that actuation of the paddle actuation control can cause axial movement of the actuation element, and axial movement of the actuation element can cause the implantable device to move between open and closed positions. The at least one visual indicator is configured to provide an indication regarding a configuration (e.g., open, closed, partially closed, other configurations, etc.) of the implantable device.

In some implementations, the paddle actuation control is a knob rotatable relative to the handle housing. In some implementations, rotation of the paddle actuation knob causes axial movement of the actuation element with respect to the handle housing and the sheath.

In some implementations, the actuation element is configured to be coupled to the implantable device.

In some implementations, a handle assembly for controlling a transvascular implantable device includes one, all, or some of the following: a handle housing, a sheath extending distally from the handle housing, an actuation element extending through the sheath, a paddle actuation control (e.g., knob, button, switch, lever, etc.) coupled to the actuation element, a paddle width adjustment element, a paddle width control (e.g., knob, button, switch, lever, etc.) coupled to the paddle width adjustment element, and an indicator (e.g., at least one visual and/or audible indicator, etc.) coupled to the paddle width adjustment element through the handle housing.

In some implementations, the actuation element is configured to be coupled to the implantable device.

In some implementations, the paddle width adjustment element is configured to be coupled to at least one of a pair of paddles of the implantable device.

In some implementations, the paddle actuation control is configured such that actuation of the paddle actuation control can cause axial movement of the actuation element, and axial movement of the actuation element can cause the implantable device to move between open and closed positions.

In some implementations, the paddle actuation control is a knob rotatable relative to the handle housing. In some implementations, rotation of the paddle actuation knob causes axial movement of the actuation element with respect to the handle housing and the sheath.

In some implementations, the paddle width control is configured such that actuation of the paddle width control can cause axial movement of the paddle width adjustment element with respect to the handle housing and the sheath, which in turn causes a width of the at least one of the pair of paddles of the implantable device to be moved from a first width to a second width or therebetween.

In some implementations, the paddle width control is configured as a paddle width control knob and rotation of the paddle width control knob causes axial movement of the paddle width adjustment element with respect to the handle housing and the sheath, which in turn causes a width of the at least one of the pair of paddles of the implantable device to be moved from a first width to a second width.

In some implementations, the at least one visual indicator is configured to provide an indication regarding one or more configurations (e.g., open, closed, partially closed, width of the paddles, other configurations, etc.) of the implantable device.

In some implementations, a steerable catheter assembly is provided. In some implementations, the steerable catheter assembly includes a catheter with at least one control element that flexes the catheter, and a control handle coupled to the catheter.

In some implementations, the control handle includes a control (e.g., knob, button, switch, lever, etc.) coupled to the at least one control element.

In some implementations, one or more visual indicators can be coupled to the at least one control element through the housing.

In some implementations, actuation of the control causes axial movement of the at least one control element with respect to the housing and moves the catheter between a first, straight configuration and a second, curved configuration.

In some implementations, the control is configured as a control knob that is rotatable with respect to a housing of the control handle. In some implementations, rotation of the control knob causes axial movement of the at least one control element with respect to the housing and moves the catheter between a first, straight configuration and a second, curved configuration.

The one or more visual indicators are configured to provide an indication regarding one or more configurations (e.g., open, closed, partially closed, width of the paddles, other configurations, etc.) of the implantable device and/or one or more configurations (e.g., straight, curved, partially curved, multiple curves, other configurations, etc.) of the catheter.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing mitral regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a perspective view of an example implantable device or implant in a closed position;

FIG. 24 illustrates an example valve repair device with paddles in an open position;

FIG. 25A illustrates another example valve repair device with paddles in a closed position;

FIG. 25B illustrates a top view of an example valve repair device;

FIG. 26 illustrates a perspective view of an example implantable device having paddles of adjustable widths;

FIG. 27 is a cross-section of the implantable device of FIG. 26 in which the implantable device is bisected;

FIG. 28 is another cross-section of the implantable device of FIG. 26 in which the implantable device is bisected along a plane perpendicular to the plane illustrated in FIG. 28;

FIG. 29 is a schematic illustration of an example implant catheter assembly coupled to an implantable device in which an actuation element is coupled to a paddle actuation control and to a driver head of the implantable device;

FIG. 30 is an illustration of the assembly of FIG. 29 with the implantable device rotated 90 degrees to show the paddle width adjustment element coupled to a movable member of the implantable device and coupled to a paddle width control;

FIG. 31 illustrates a distal end of an example delivery assembly including a delivery apparatus and an implantable device;

FIG. 32 illustrates a proximal end of the example delivery assembly of FIG. 31;

FIG. 33A illustrates a cross-section of the handle of the steerable catheter assembly of the example delivery assembly of FIG. 32;

FIG. 33B illustrates an enlarged portion of FIG. 33A as indicated by the box 33B in FIG. 33A;

FIG. 34 is a perspective view of an example handle of an implant catheter assembly;

FIG. 35 is a cross-section of the handle of FIG. 34 perpendicular to the plane defined by line A-A, in which one of the clasp control tubes is bisected;

FIG. 36 is another cross-section of the handle of FIG. 34 perpendicular to the plane defined by line B-B, in which the release knob is bisected;

FIG. 37 is an enlarged portion of FIG. 36;

FIG. 38 is a perspective view of an example handle of an implant catheter assembly including a visual indicator;

FIG. 39 is a side view of the example handle of FIG. 38;

FIG. 40A is an example cross-sectional view of the example handle of FIG. 39 in which the visual indicator is configured to provide an indication regarding a configuration of the device;

FIG. 40B is an example cross-sectional view of the example handle of FIG. 39 in which the visual indicator is configured to provide an indication regarding a width of the paddles of the device;

FIG. 41 is another example handle of an implant catheter assembly including a visual indicator;

FIG. 42 is an exploded view of an example paddle width control including a visual indicator;

FIG. 43 is a side view of an example paddle width control including a visual indicator;

FIG. 44 is a cross-sectional view of the example paddle width control of FIG. 43;

FIG. 45 is a perspective view of another example handle of an implant catheter assembly including two visual indicators; and

FIG. 46 is a perspective view of another example handle of an implant catheter assembly including two visual indicators and providing additional detail regarding the width control mechanism.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a non-living simulation, such as on a cadaver, cadaver heart, simulator (e.g., with the body parts, tissue, etc. being simulated), etc. The term simulation encompasses use on cadaver, computer simulator, imaginary person (e.g., demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

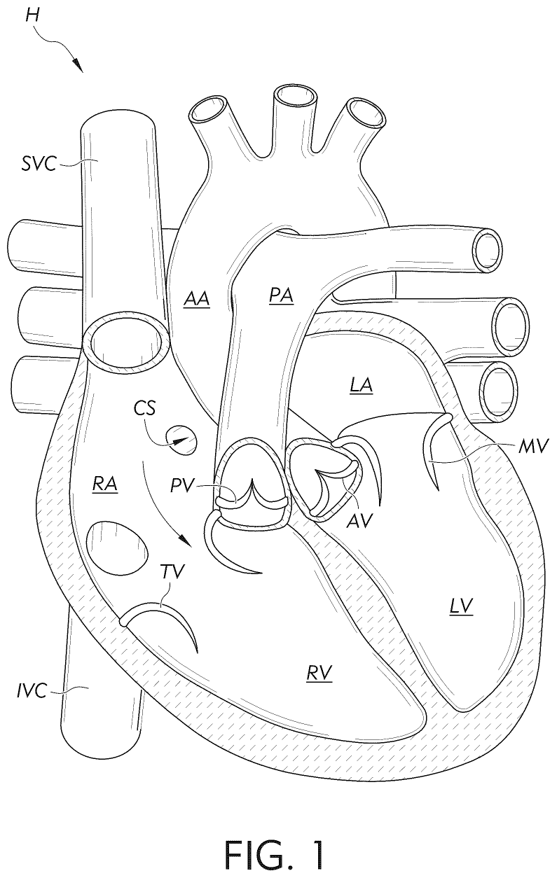

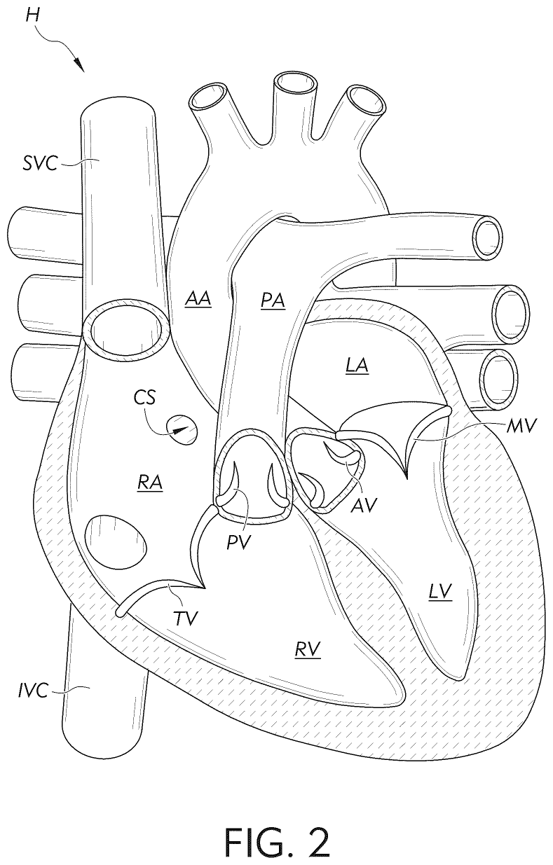

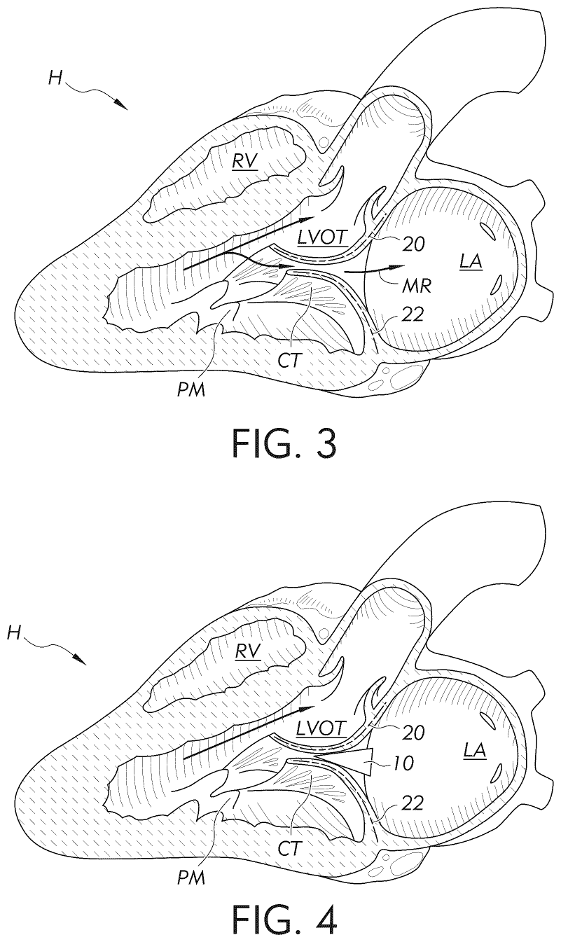

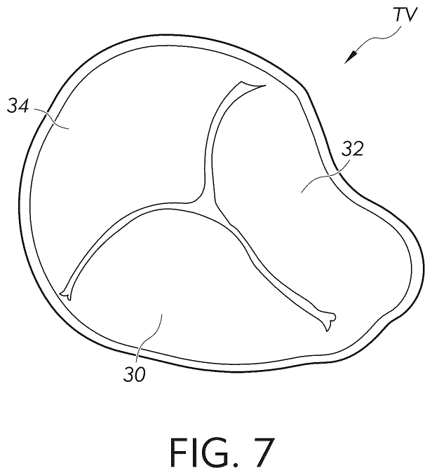

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

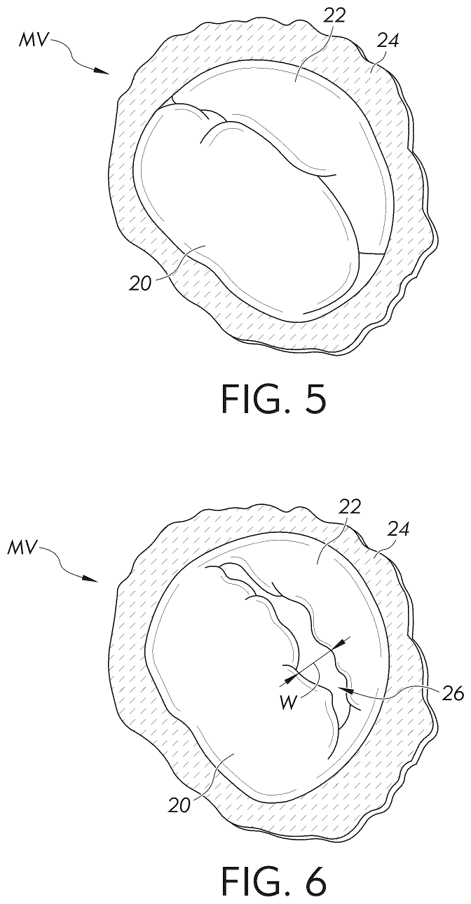

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of an implantable device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaption element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element. The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT Patent Application Publication No. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

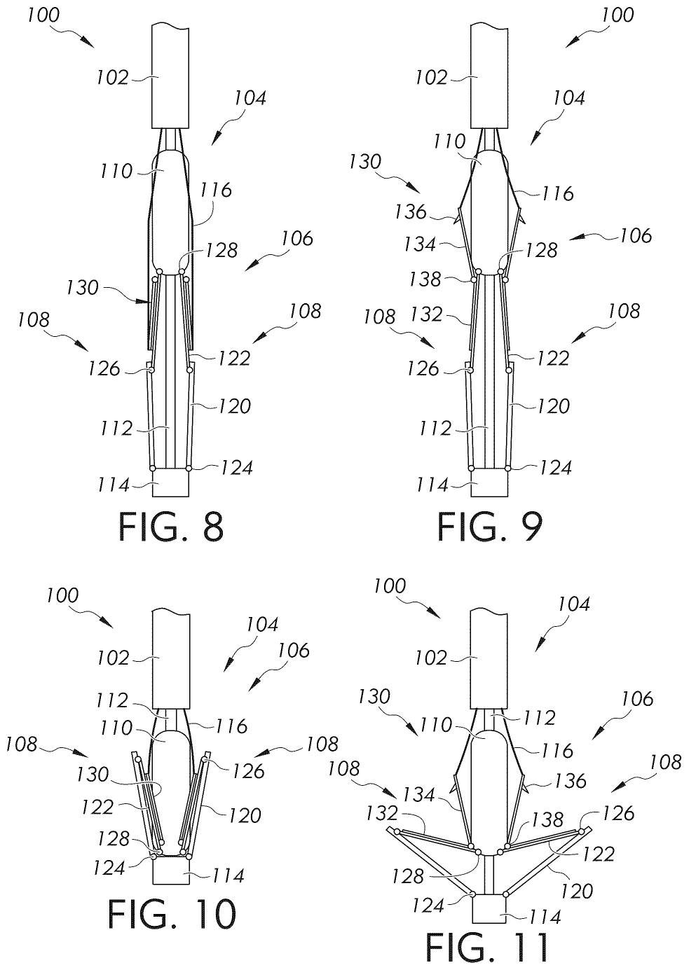

Referring now to FIGS. 8-15, a schematically illustrated implantable device or implant 100 (e.g., an implantable prosthetic device, a prosthetic spacer device, a valve repair device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for an implantable device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, actuation shaft, actuation tube, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112. These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., coupler, fastener, lines, such as sutures, etc.) removably attaches the spacer or coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., clasps, clamps, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the barbs, friction-enhancing elements, or other securing structures 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other optional friction-enhancing elements 136 and pinching the leaflets between the moveable and fixed arms 134, 132. The friction-enhancing elements 136 (e.g., barbs, protrusions, ridges, grooves, textured surfaces, adhesive, etc.) of the clasps 130 increase friction with the leaflets or can be configured to partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable portions 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or other gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element and cap (or other attachment portion) are used to control one paddle, and the other independent actuation element and cap (or other attachment portion) are used to control the other paddle.

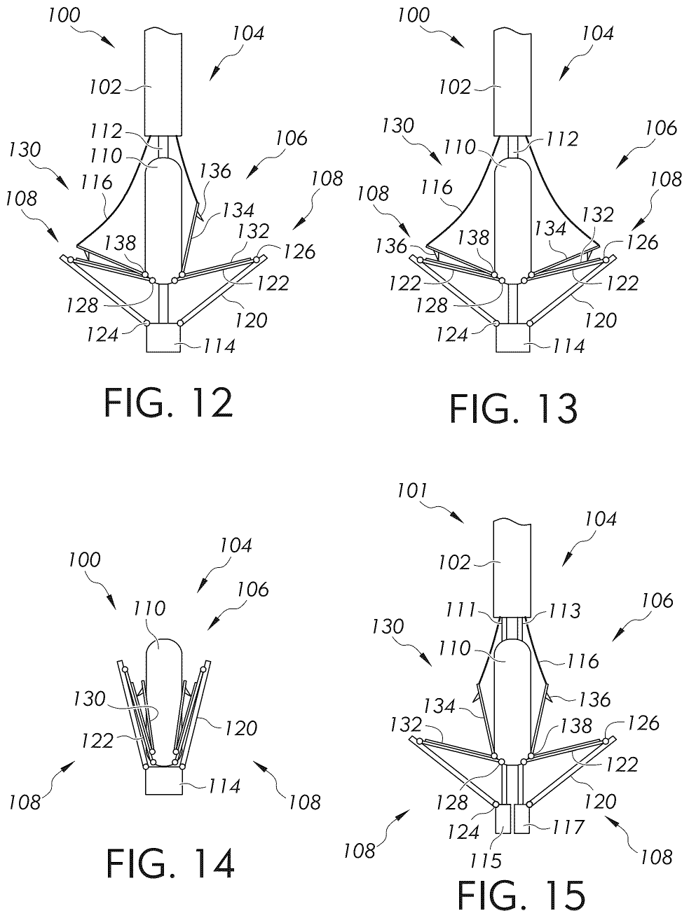

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

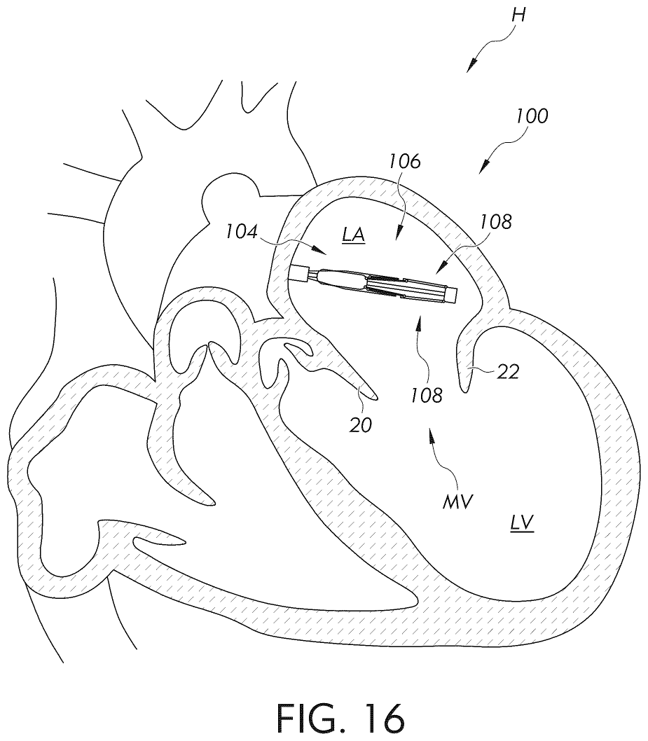

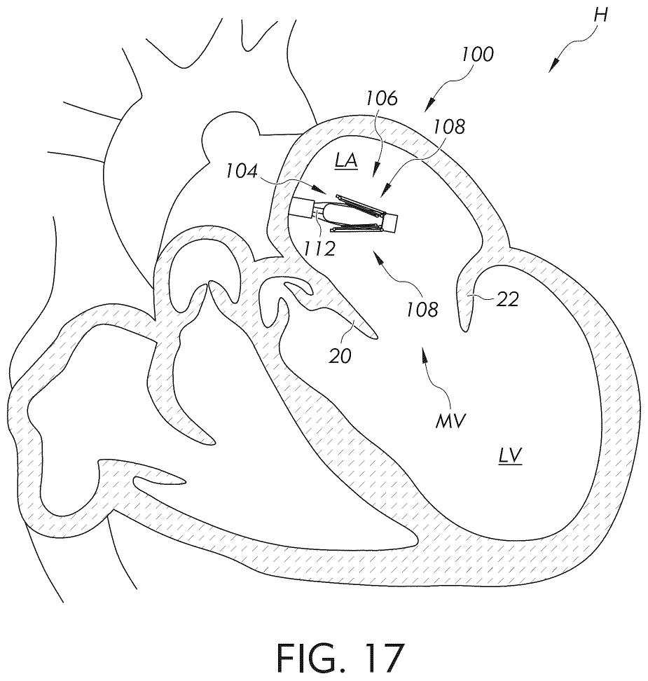

Referring now to FIGS. 16-21, the implantable device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

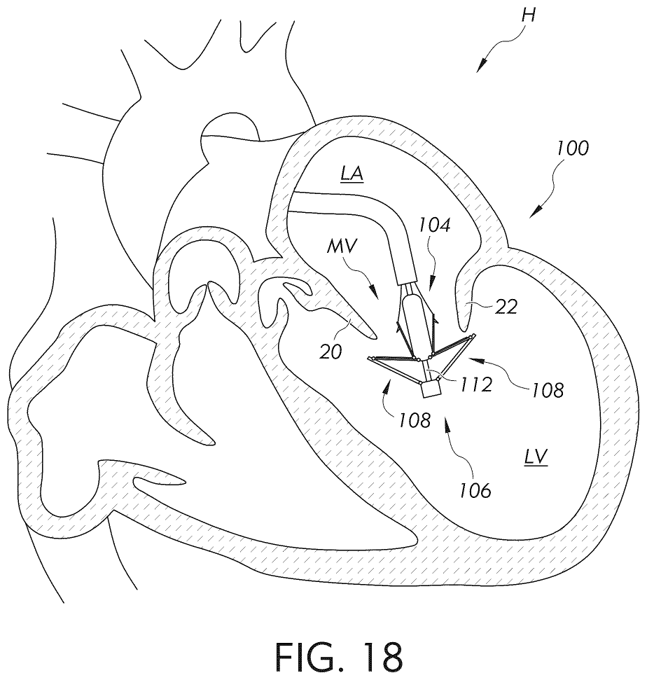

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

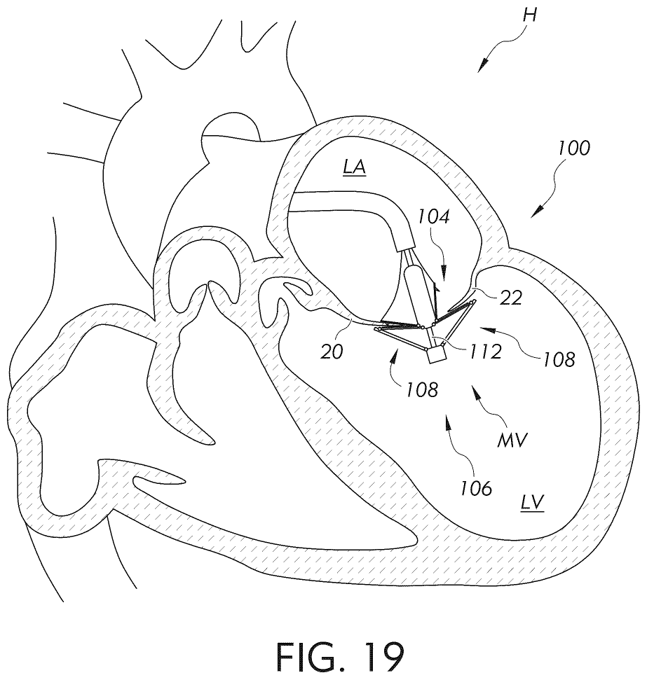

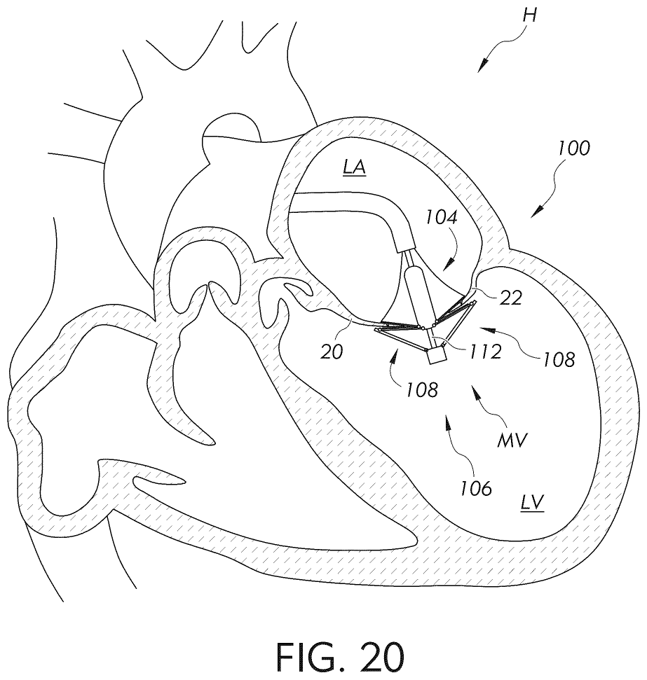

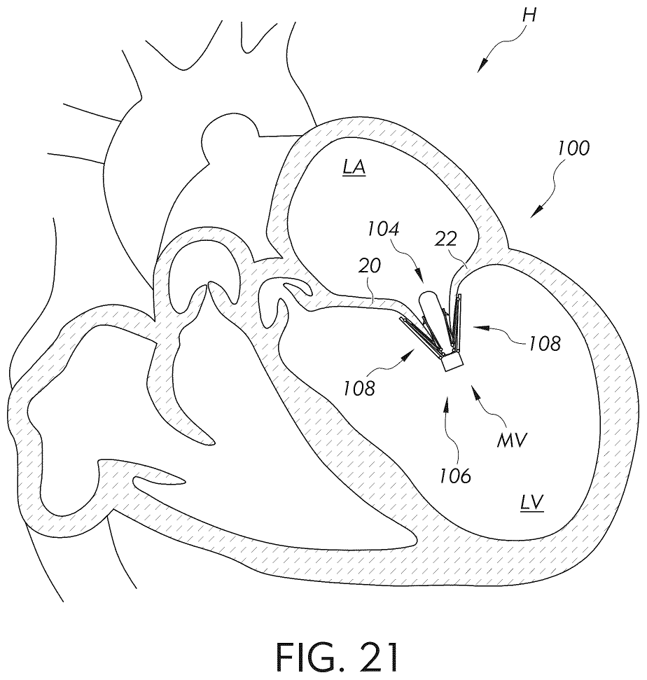

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

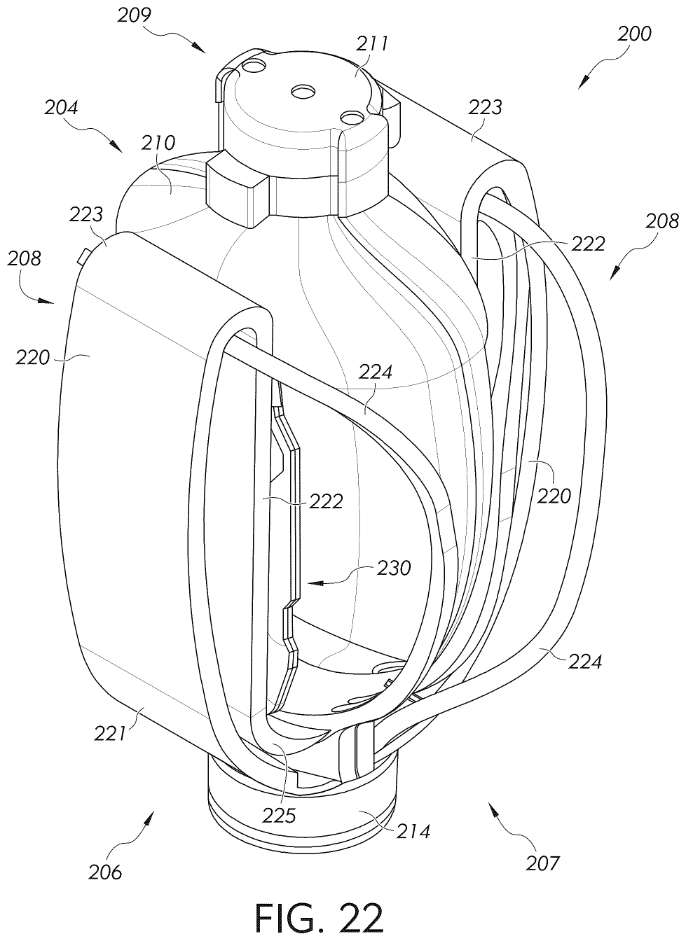

Any of the features disclosed by the present application can be used in a wide variety of different valve repair devices. FIGS. 22-24 illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 8-24.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

In some implementations, the implantable device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism (e.g., coupler, fastener, clamp, lines or sutures, etc.) of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the implantable device or implant 200. In some implementations, the actuation element extends through the proximal collar 211, and coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member (e.g., bar, plate, rod, etc.) or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety. The method(s) in these references can be adapted for use with the concepts herein and be performed on a living animal or on a simulation, such as on a cadaver, cadaver heart, simulator (e.g., with the body parts, heart, tissue, etc. being simulated), etc. mutatis mutandis.

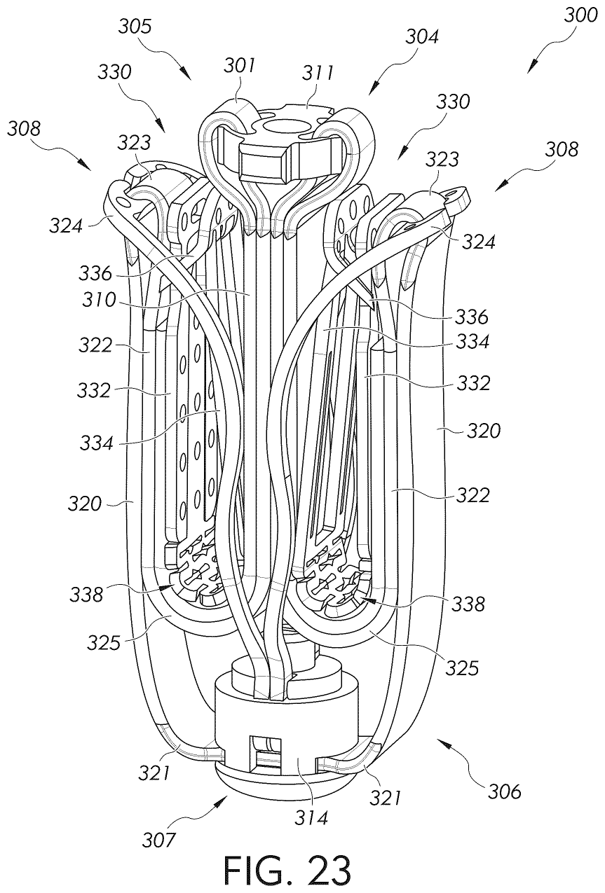

Referring now to FIG. 23, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, friction-enhancing elements 336 (e.g., barbs, ridges, protrusions, textured surfaces, etc.), and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety. The method(s) in these references can be adapted for use with the concepts herein and be performed on a living animal or on a simulation, such as on a cadaver, cadaver heart, simulator (e.g., with the body parts, heart, tissue, etc. being simulated), etc. mutatis mutandis.

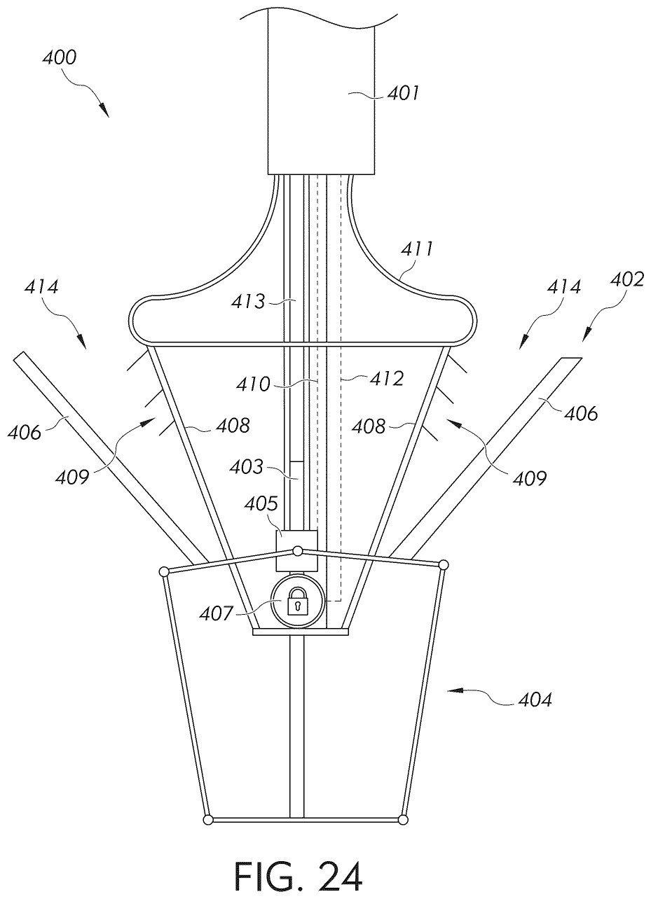

FIG. 24 illustrates another example of one of the many valve repair systems 400 for repairing a native valve of a patient that the concepts of the present application can be applied to. The valve repair system 400 includes a delivery device 401 and a valve repair device 402.

The valve repair device 402 includes a base assembly 404, a pair of paddles 406, and a pair of gripping members 408. In one example, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the valve repair device 402 is attached to the valve tissue. When the paddles 406 are in the closed position, the paddles engage the gripping members 408, such that, when valve tissue is attached to the optional barbed portion 409 of the gripping members, the paddles secure the valve repair device 402 to the valve tissue. In some implementations, the gripping members 408 are configured to engage the paddles 406 such that the optional barbed portion 409 engages the valve tissue and the paddles 406 to secure the valve repair device 402 to the valve tissue. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410 (e.g., wire, rod, shaft, tube, etc.), a gripper control mechanism 411 (e.g., line, suture, wire, etc.), and a lock control mechanism 412 (e.g., line, suture, wire, rod, etc.). The paddle control mechanism 410 is mechanically attached to the coupler 405 to move the coupler along the shaft, which causes the paddles 406 to move between the open and closed positions. The paddle control mechanism 410 can take any suitable form, such as, for example, a shaft or rod. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, etc.

The lock control mechanism 412 is configured to lock and unlock the lock. The lock 407 locks the coupler 405 in a stationary position with respect to the shaft 403 and can take a wide variety of different forms and the type of lock control mechanism 412 may be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The valve repair device 402 is movable from an open position to a closed position. The base assembly 404 includes links that are moved by the coupler 405. The coupler 405 is movably attached to the shaft 403. In order to move the valve repair device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the valve repair device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the valve repair device 402 in the closed position. After the valve repair device 402 is maintained in the locked condition by the lock 407, the valve repair device 402 is removed from the delivery device 401 by disconnecting the shaft 403 from the placement shaft 413. In addition, the valve repair device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety. The method(s) in these references can be adapted for use with the concepts herein and be performed on a living animal or on a simulation, such as on a cadaver, cadaver heart, simulator (e.g., with the body parts, heart, tissue, etc. being simulated), etc. mutatis mutandis.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

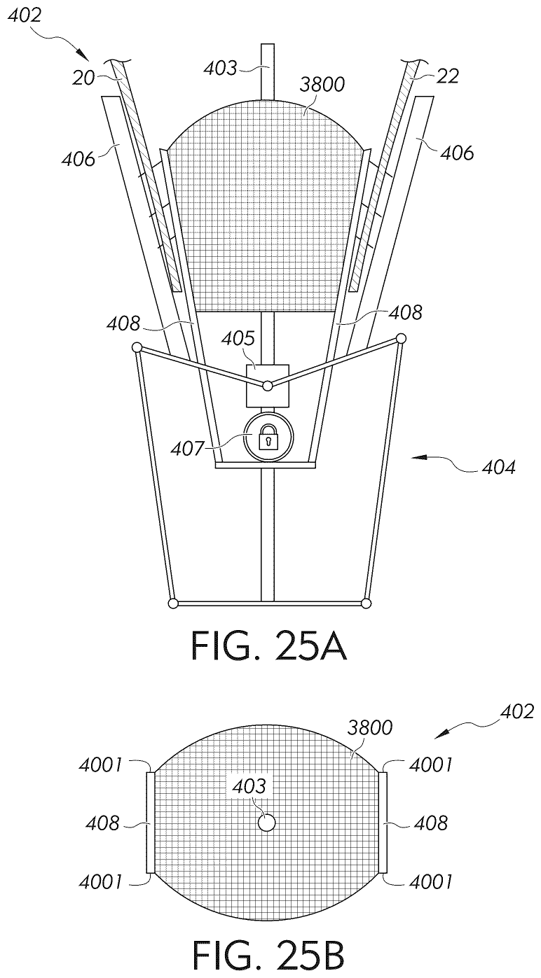

Referring to FIGS. 25A-25B, an example implementation of a valve repair device 402 has a coaptation element 3800. The valve repair device 402 can have the same configuration as the valve repair device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the valve repair device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the valve repair device 402 is attached to the leaflets.

The coaptation element 3800 can be disposed around one or more of the shafts or other control elements of the valve repair system 400. For example, the coaptation element 3800 can be disposed around the shaft 403, the shaft 413, the paddle control mechanism 410, and/or the lock control mechanism 412.

The valve repair device 402 can include any other features for a valve repair device discussed in the present application, and the valve repair device 402 can be positioned to engage valve tissue as part of any suitable valve repair system (e.g., any valve repair system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). The method(s) in these references can be adapted for use with the concepts herein and be performed on a living animal or on a simulation, such as on a cadaver, cadaver heart, simulator (e.g., with the body parts, heart, tissue, etc. being simulated), etc. mutatis mutandis.

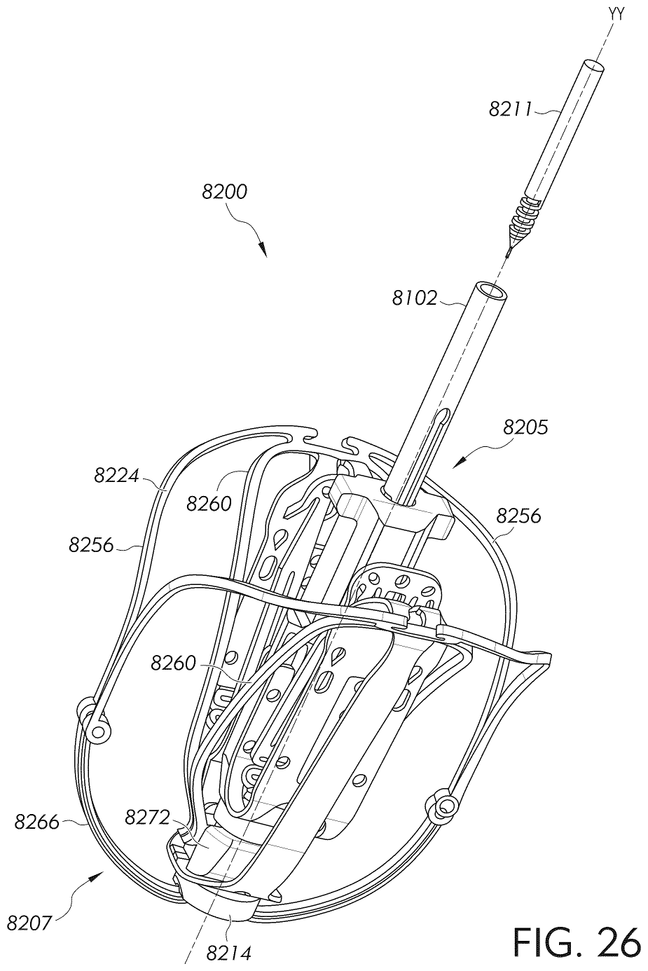

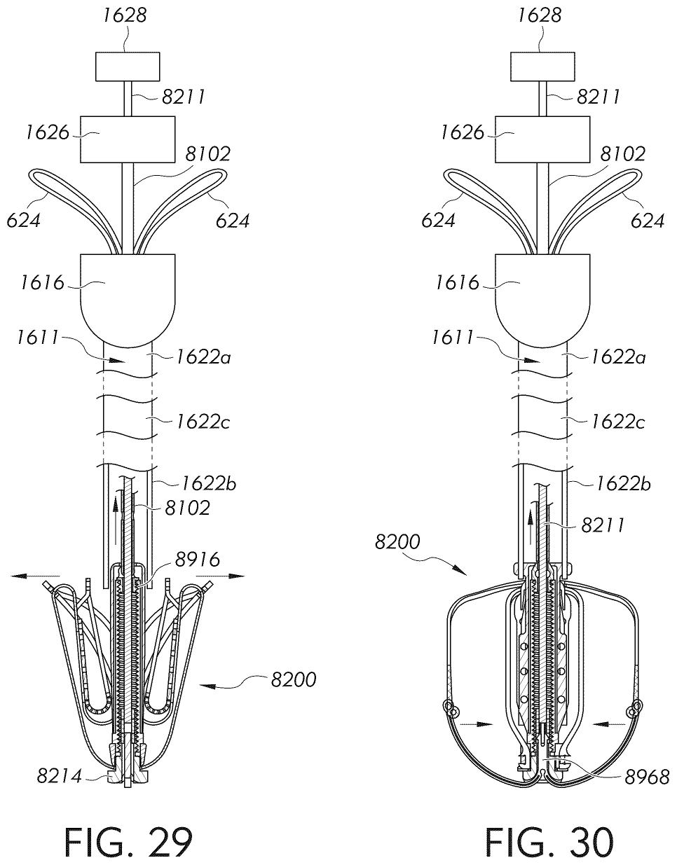

FIGS. 26-30 illustrate another example of one of the many valve repair systems for repairing a native valve of a patient that the concepts of the present application can be applied to. Referring to FIGS. 29 and 30, the valve repair system includes an implant catheter assembly 1611 and an implantable valve repair device 8200. Referring to FIGS. 26-28, the implantable device 8200 includes a proximal or attachment portion 8205, paddle frames 8224, and a distal portion 8207. The proximal portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the proximal portion 8205, the outer frame portions 8256 form a curved shape. In the illustrated example, the shape of the outer frame portions 8256 resembles an apple shape in which the outer frame portions 8256 are wider toward the proximal portion 8205 and narrower toward the distal portion 8207. In some implementations, however, the outer frame portions 8256 can be otherwise shaped.

The inner frame portions 8260 extend from the proximal portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

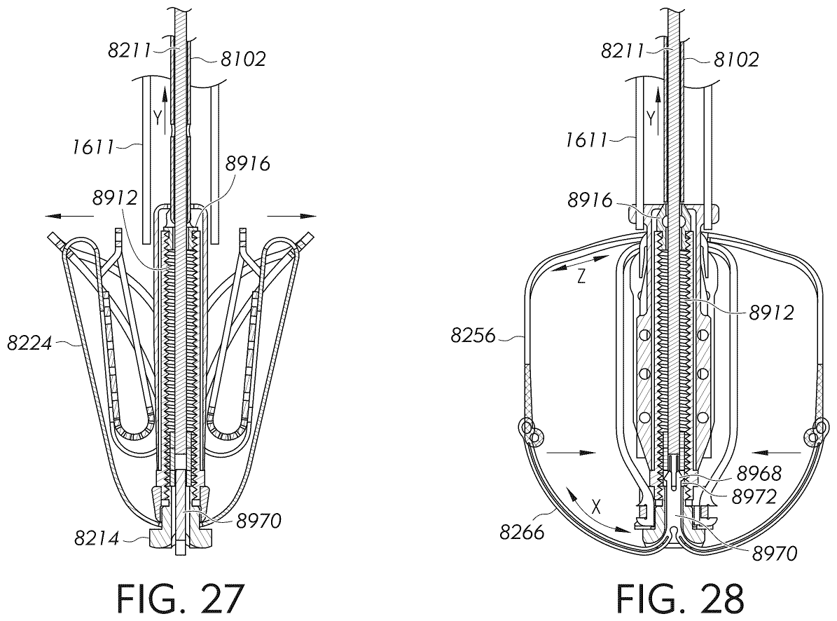

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions. When the coupler 8972 is attached to the width adjustment element 8211, the coupler is released from the receiver. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the implantable device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as disclosed above.

In the illustrated example, a driver head 8916 is disposed at a proximal end of the actuation element 8102. The driver head 8916 releasably couples the opening/closing actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an implementation of an implant catheter assembly 1611 in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the implant catheter assembly 1611 can be coupled to the handle 1616, and a distal end portion 1622b of the shaft or catheter can be coupled to the implantable device 8200. The actuation element 8102 can extend distally from the paddle actuation control 1626, through the handle 1616, through the shaft or catheter of the implant catheter assembly 1611, and through the proximal end of the device 8200, where it couples with the driver head 8916. The actuation element 8102 can be axially movable relative to the outer shaft of the implant catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the implant catheter assembly 1611 is an elongate shaft extending axially between the proximal end portion 1622a, which is coupled to the handle 1616, and the distal end portion 1622b, which is coupled to the device 8200. The outer shaft of the implant catheter assembly 1611 can also include an intermediate portion 1622c disposed between the proximal and distal end portions 1622a, 1622b.

In some implementations, a delivery apparatus is configured to move the implantable device or implant between its various configurations and/or to implant the implantable device or implant in the native heart valve. For example, controls on a handle used in a delivery assembly can be configured to enable improved control of the implantable device or implant, as will be described.

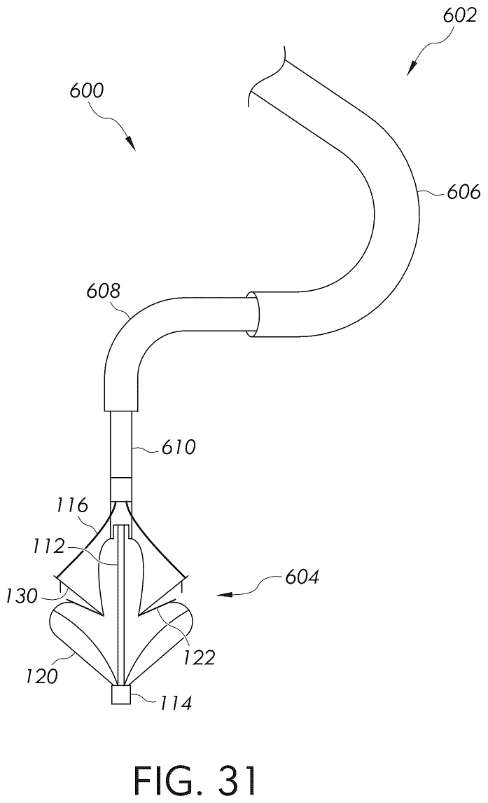

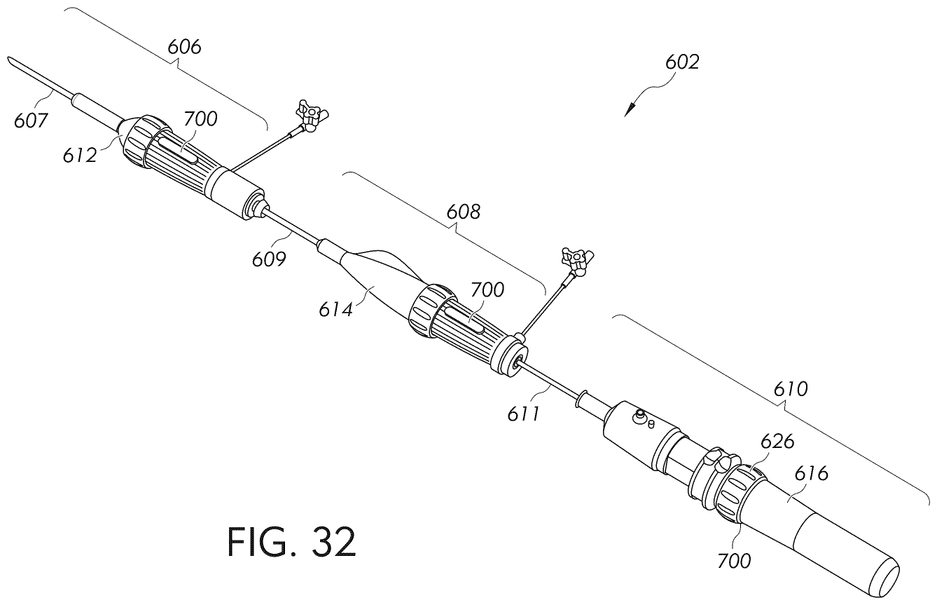

FIGS. 31-32 show an implementation of a delivery assembly 600 and its components. Referring to FIG. 31, the delivery assembly 600 can comprise the delivery apparatus 602 and an implantable device 604. The delivery apparatus 602 can comprise a plurality of catheter assemblies and optional catheter stabilizers (not shown in FIGS. 31 and 32). For example, in the illustrated example, the delivery apparatus 602 includes a delivery catheter assembly 606, a steerable catheter assembly 608, and an implant catheter assembly 610. The steerable catheter assembly 608 extends coaxially through the delivery catheter assembly 606, and the implant catheter assembly 610 extends coaxially through the steerable catheter assembly 608 and the delivery catheter assembly 606. The implantable device 604 can be releasably coupled to a distal portion of the implant catheter assembly 610, as further described below. It should be appreciated that the implantable device 604 can be any device described herein.

As shown in FIG. 32, each of the delivery catheter assembly 606, the steerable catheter assembly 608, and the implant catheter assembly 610 includes a sheath or shaft 607, 609, 611 extending from a handle 612, 614, 616, respectively. The handles 612, 614, 616 are located at a proximal end of each of the corresponding sheaths or shafts, and include one or more control members (e.g., knob, slider, switch, lever, etc.) to enable a user to manipulate the catheter assembly (e.g., bend or rotate the sheath or shaft of the catheter assembly) and/or control a component coupled to the corresponding catheter assembly (e.g., a control wire extending through the shaft of the catheter assembly).

The delivery catheter assembly 606 and the steerable catheter assembly 608 can be used, for example, to access an implantation location (e.g., a native mitral valve region of a heart) and/or to position the implant catheter assembly 610 at the implantation location. Accordingly, in some implementations, the delivery catheter assembly 606 and the steerable catheter assembly 608 are configured to be steerable. Features of catheter assemblies disclosed by U.S. Pat. No. 10,653,862 and U.S. Pat. No. 10,646,342 can be used in the catheter assemblies 606, 608, 610. U.S. Pat. Nos. 10,653,862 and 10,646,342 are hereby incorporated by reference in their entireties.

In some implementations, one or more of the handles 612, 614, and 616 can incorporate a visual and/or audible indicator 700 configured to provide an indication regarding a position of a corresponding sheath or shaft 607, 609, 611 and/or a configuration of the implantable device 604 (e.g., an implantable valve repair device, an annuloplasty ring, a replacement valve, a chordae repair or replacement device, an anchor configured to be implanted in the heart, etc.), as will be described in greater detail below by way of more specific examples. In some implementations, the visual indicator 700 can be positioned on a catheter handle and/or be configured to provide an indication regarding a position of a sheath and/or a shaft used to deliver an implantable valve repair device, an annuloplasty ring, a replacement valve, a chordae repair or replacement device, an anchor configured to be implanted in the heart, etc. and/or a configuration of an implantable valve repair device, an annuloplasty ring, a replacement valve, a chordae repair or replacement device, an anchor configured to be implanted in the heart, etc.

Although the implementation in FIG. 32 includes a visual indicator 700 on each of the handles 612, 614, and 616, it is contemplated that in some implementations, fewer than all of the handles 612, 614, and 616 can include a visual indicator 700. Moreover, it is contemplated that one or more of the visual indicators can be replaced with an auditory indicator, such as a speaker configured to emit a sound indicative of a position of the corresponding sheath or shaft 607, 609, 611, or a configuration of the implantable device 604. Accordingly, in any one of the implementations described herein, the visual indicator 700, including LED lights, LED displays, LCD displays or the like included in the visual indicator, can be replaced or supplemented with an auditory indicator. In some implementations, the visual and/or audible indicator 700 provide an indication regarding a position of a corresponding sheath or shaft 607, 609, 611 and/or a configuration of the implantable device 604 by changing the intensity, color, etc. of light emitted by the indicator and/or by increasing or changing the sound emitted by the indicator.

In FIG. 32, the visual indicator 700 of handles 612 and 614 is configured to provide an indication regarding the flex of the corresponding shaft or sheath 607, 609 coupled to the handle 612, 614. The visual indicator 700 can light up different colors (e.g., red, blue, yellow, etc.) or different intensities (e.g., dim, moderate, bright, etc.) to indicate an amount of flex in the corresponding shaft or sheath 607, 609. In some implementations, the visual indicator 700 can be in the form of a light emitting diode (LED) readout, touchscreen, or other visual display configured to display an alphanumeric description (e.g., 1-10, a specific degree of flex, or “no flex,” “partially flexed,” or “fully flexed”). In some implementations in which the visual indicator 700 reports a specific degree of flex, it is contemplated that the reported degree of flex can be in 5° increments to account for system limitations that may result from friction between the shafts and sheaths 607, 609, 611, manufacturing variances, and other system components.

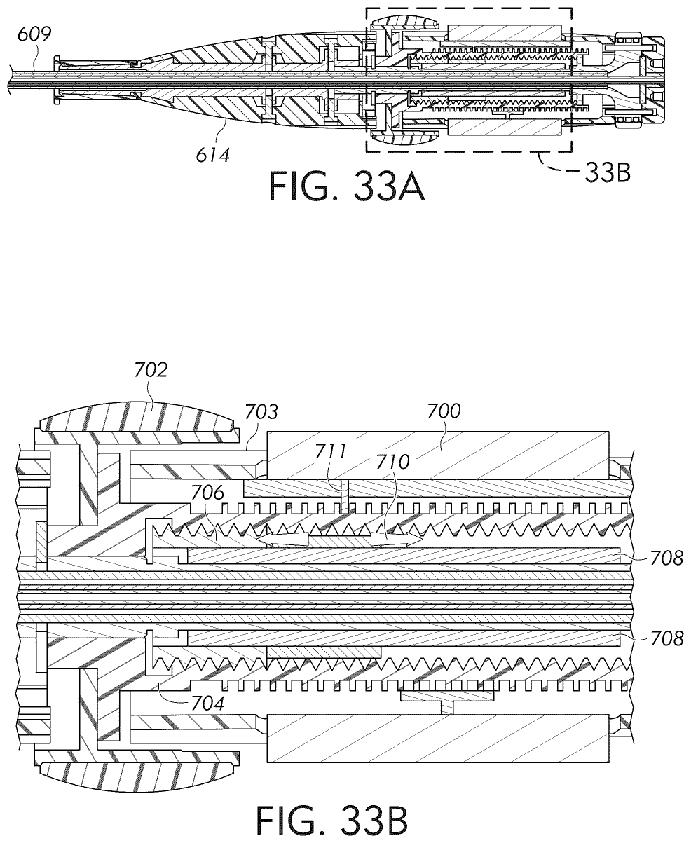

FIGS. 33A and 33B show a cross-section of the handle 614 of the steerable catheter assembly 608 shown in FIG. 32. The steerable catheter assembly 608 includes a control 702. The control 702 can be configured in a variety of ways, e.g., as a knob, button, switch, lever, gear, worm screw, combination of these and/or other components, etc.

In some implementations, control 702 (which are described and/or depicted herein as knob 702, though it should be understood that a knob is only one control configuration that can be used) is actuated by being rotated with respect to the housing 703 of the handle 614 (other actuation methods besides rotation can be used in some implementations). In some implementations, the knob 702 is fixedly coupled to an internally threaded tube 704 positioned within the housing 703. When the knob 702 is rotated about the axis of the handle 614, the internally threaded tube 704 rotates with respect to the housing 703. In some implementations, an externally threaded retractor 706 is positioned within and engaged with the internally threaded tube 704. The externally threaded retractor 706 is rotationally fixed with respect to the housing 703. The externally threaded retractor 706 can be rotationally fixed in a wide variety of different ways.

In the example illustrated in FIGS. 33A and 33B, a pair of guide rods 708 extends axially within the handle 614, and each of the pair of guide rods 708 is fixed to the housing 703 at both ends of the guide rod 708. Accordingly, as the internally threaded tube 704 is rotated, the externally threaded retractor 706 is advanced linearly in an axial direction (i.e., distally and proximally) along the pair of guide rods 708.

In some implementations, the externally threaded retractor 706 is coupled to a control element (not shown), such as a wire, a tube, a shaft, etc., such that linear movement of the externally threaded retractor 706 causes linear movement of the control element. The control element is fixedly connected at a distal end to the sheath or shaft 609. In some implementations, the control element is coupled to a ring that is affixed within the sheath or shaft 609. In some implementations, the control element is coupled directly to an internal surface of the sheath or shaft 609. The axial movement of the control element moves the sheath or shaft 609 between a first, straight configuration, and a second, curved configuration.

In some implementations, the internally threaded tube 704 is also coupled to a switch, sensor, etc., such as the illustrated slider 710 and sensor 711. The slider 710 and sensor 711 can take a wide variety of different forms. For example, the slider 710 can be a magnet, material with distinct optical properties, material with distinct acoustic properties, or any other material or configuration that can be sensed by the sensor 711. The sensor 711 can be a hall sensor, an optical sensor, an ultrasonic sensor, a proximity sensor, etc. One or both of the slider 710 and the sensor 711 can be annular, so that the sensor is in sensing proximity to the slider regardless of the rotational position of the knob 702.

In some implementations, the slider 710 is electronically coupled to the visual indicator 700. In some implementations, the sensor 711 can be coupled to the visual indicator 700 in a variety of different ways, such as through a microprocessor (not shown). Accordingly, as the knob 702 and attached threaded tube 704 are rotated, the threaded retractor 706 and the slider 710 are moved in the axial direction (i.e., distally and proximally). The sensor 711 senses the movement of the slider and stimulates the visual indicator 700. In the example illustrated in FIGS. 33A and 33B, the visual indicator 700 is an LED indicator configured to emit light at varying intensities. In this implementation, the axial position of the slider 710 corresponds to the intensity of the LED of the visual indicator 700.

As a result of the knob 702 being coupled to both the visual indicator 700 and the control element (e.g., shaft, rod, tube, etc.) through the internally threaded tube 704 and threaded retractor 706, the rotation of the internally threaded tube 704 can be an effective indicator of the amount of flex of the shaft or sheath 609. For example, the shaft or sheath 609 can be in a first, straight configuration with the externally threaded retractor 706 coupled to the control positioned distally within the housing 703. As the knob 702 is rotated with respect to the housing 703, the internally threaded tube 704 rotates, thereby driving both the externally threaded retractor 706 and the slider 710 in the proximal direction. The proximal movement of the externally threaded retractor 706 creates tension on the control element, pulling the control element, and causing the shaft or sheath 609 to flex. The more the internally threaded tube 704 rotates, the more proximally the externally threaded retractor 706 and the slider 710 are driven, and the greater the flex in the shaft or sheath 609.

Although the implementation in FIGS. 33A and 33B are described with respect to handle 614, it is contemplated that the slider 710, sensor 711, and visual indicator 700 can be incorporated into other handles, such as handle 612, in the same or a similar manner. Furthermore, it is contemplated that other activation mechanisms can be employed as an alternative to the slider 710, sensor 711, and/or the LED indicator. For example, other switches, controls, sensors, and/or actuation devices can be incorporated into the handle.

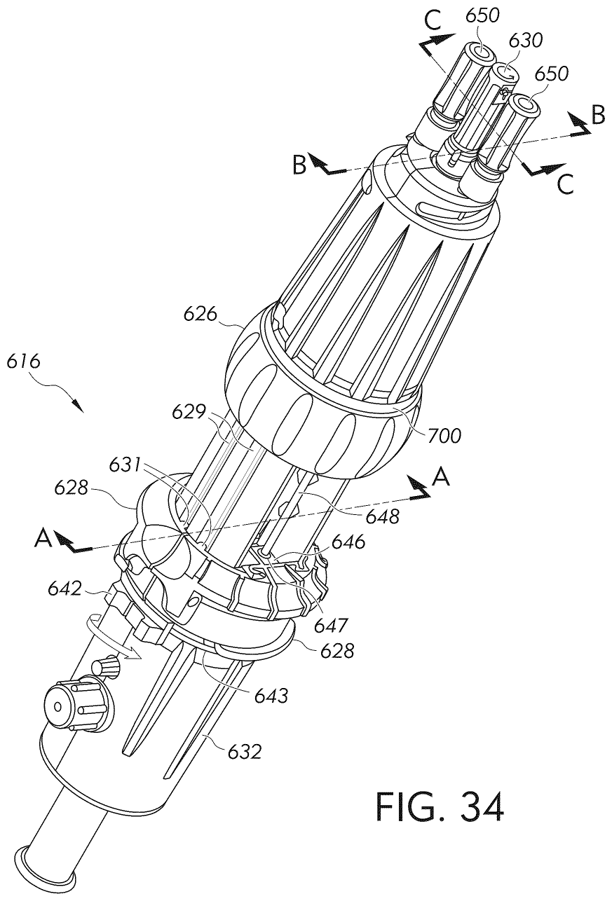

In some implementations, as shown for example in FIG. 32, the handle 616 of the implant catheter assembly 610 also includes a visual indicator 700. As shown in FIGS. 32 and 34, the visual indicator 700 on the handle 616 can be configured in the form of a ring adjacent to the control 626 (which can be configured as a knob that is actuated by rotation in the examples depicted, but can be configured as another type of control and actuated in other ways), and be configured to provide an indication regarding a location, condition, etc. of the paddles of the implantable device 604. However, in some implementations, the visual indicator 700 can be in the form of one or more light emitting diodes (LEDs), a light emitting diode (LED) readout, touchscreen, or other visual display configured to display an alphanumeric description (e.g., 1-10 or “elongated,” “open,” or “closed”).

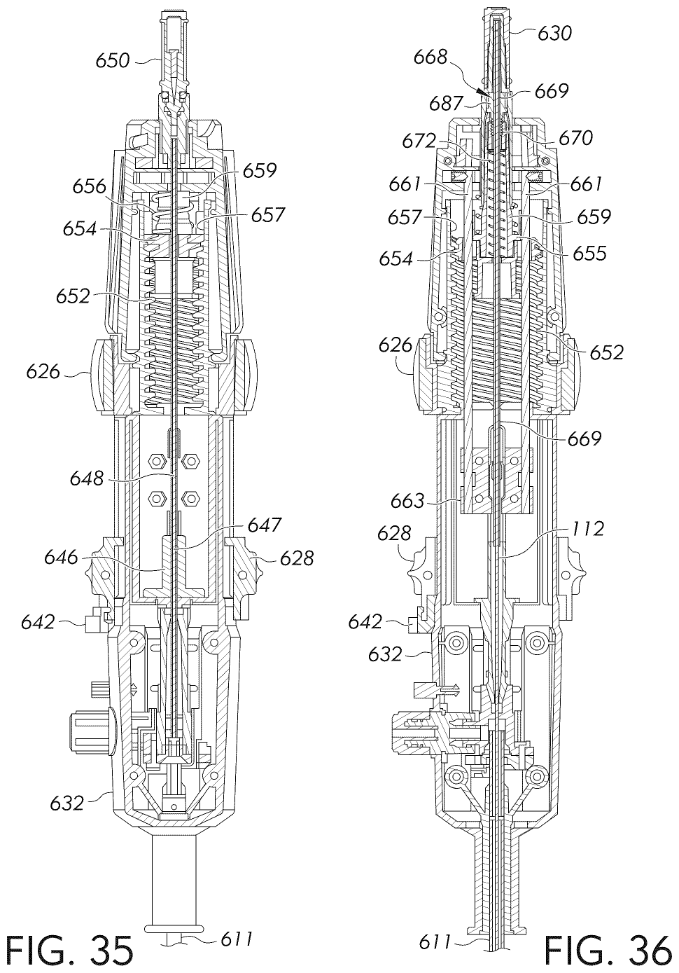

In the example shown in FIG. 34, the handle 616 includes a housing 632 to which the various controls for delivering the implantable device 604 are coupled. The housing 632 can be formed from various materials, including polymers such as polycarbonate, and can be formed as a unitary body (e.g., through injection molding) or fastened together in any one of a variety of manners, including fasteners, pins, adhesives, or the like. As shown in FIGS. 35-37, the housing 632 comprises a plurality of lumens, through which the clasp actuation lines 624 and the actuation element 112 extend.

In some implementations, as shown in FIGS. 34-36, the housing 632 includes a slide lock 642 that is configured to slide between a first position, in which the slide lock 642 is coupled to one of the clasp control members 628 (e.g., slider, etc.), to a second position, in which the slide lock 642 is coupled to both of the clasp control members 628. For example, each of the clasp control members 628 can include a flange 643 to which the slide lock 642 is slidably coupled. Accordingly, the slide lock 642 can be moved to the first position (as indicated by the arrow shown in FIG. 34) in which the slide lock is engaged with the flange 643 of one of the clasp control members 628 (e.g., to enable independent movement of each clasp actuation line relative to the other) and the second position (shown in FIG. 34) in which the slide lock 642 is engaged with the flange 643 of both of the clasp control members 628 (e.g., to enable simultaneous movement of the clasp actuation lines) in order to selectively control relative movement between the clasp control members 628. When the slide lock is in the first position, each of the clasp control members is axially movable independently of the other clasp control member, and when the slide lock is in the second position, the clasp control members are axially movable together. Although not shown in the figures, in some implementations, each flange 643 can include a stop to limit the position of the slide lock 642 along the flange 643.

In some implementations, such as the example illustrated by FIGS. 34 and 35, the coupler 646 includes at least one aperture 647, in which the clasp control tube 648 is connected. The clasp control member 628 is slidably coupled to the housing 632, such as by the mating channels 629 of the housing 632 and the projections 631 of the clasp control member 628 (see FIG. 34) and/or by the clasp control tube 648 extending through a guide passage formed in the housing (not shown). The clasp control member, the coupler 646, and the clasp control tube 648 can slide together axially (i.e., distally and proximally) with respect to the housing 632 to open and close the clasp. Only a single coupler 646 and clasp control tube 648 can be seen in FIGS. 34 and 35. The clasp control tube 648 can be fixed at a proximal end to a suture lock 650 that is used to secure the clasp actuation line 624 to the clasp control tube 648.

Returning to FIG. 35, as the clasp control member 628 is moved in a proximal direction, the clasp control tube 648 also moves in the proximal direction, pushing the suture lock 650 in the proximal direction with respect to the handle 616. As the suture lock 650 moves in the proximal direction, the loop of the clasp actuation line 624 extending from the distal end of the outer shaft 611 moves proximally, pulling the clasp 130. To release the clasp 130, such as to grasp the leaflet, the clasp control member 628 is moved in a distal direction, which in turn moves the clasp control tube 648 and suture lock 650 in the distal direction, thereby moving the loop of the clasp actuation line 624 in the distal direction, enabling the clasp 130 to move toward the inner paddles 122, grasping the leaflet between the clasp 130 and the inner paddle 122.

As previously mentioned, in some implementations, the handle 616 further comprises a control 626 (which can be, for example, as a knob that is actuated by rotation, but can be configured in other ways and actuated in other ways as well). In some implementations, control 626 is configured to rotate about an axis of the handle 616 and to control the position of the actuation element 112 relative to the handle 616 and outer shaft 611; however, control 626 can be configured to be actuated in other ways as well, e.g., pushing, switching, swiping, etc.

In some implementations, as can be seen in FIGS. 35-37, the knob 626 can be fixedly coupled to an internally threaded tube 652 positioned within the housing 632 of the handle 616. When the knob 626 is rotated about the axis of the handle 616, the internally threaded tube 652 rotates with respect to the housing 632. An externally threaded nut or retractor 654 is positioned within and engaged with the internally threaded tube 652. The externally threaded nut or retractor 654 is rotationally fixed with respect to the housing 632. The externally threaded nut or retractor 654 can be rotationally fixed in a wide variety of different ways. In the example illustrated by FIG. 36, a pair of guide rods 661 extend axially within the handle 616, and each of the pair of guide rods 661 is fixed to the housing 632 at both ends of the guide rod 661. A mounting bracket 663 couples the pair of guide rods 661 to the housing 632 at the distal end of each of the pair of guide rods 661. Accordingly, as the internally threaded tube 652 is rotated, the externally threaded retractor 654 is advanced linearly in an axial direction (i.e., distally and proximally) along the pair of guide rods 661.

In some implementations, the externally threaded retractor 654 is connected to the actuation element 112 such that linear movement of the externally threaded retractor 654 causes linear movement of the actuation element 112, thereby moving the device between a fully elongated configuration, an open configuration, and a closed configuration. The translation of the rotational movement of the knob 626 into linear motion of the actuation element 112 can result in improved control and precision, thereby leading to improved precision during the opening and closing of the device.

In some implementations, the internally threaded tube 652 includes an unthreaded portion 657. In some implementations, a clutch spring 656 is positioned around an unthreaded proximal portion 659 of the retractor 654. The clutch spring presses against a proximal end surface 655 of the threaded portion of the retractor 654. Proximal movement of the actuation element 112 after closure of the device 604 can result in overclosure or compression of the valve repair device. The position of the unthreaded portion 657 is selected to prevent or inhibit over-retraction of the actuation element 112 and thereby prevent or inhibit over-closing of the valve repair device. That is, the externally threaded retractor 654 is no longer driven proximally when the externally threaded nut or retractor 654 reaches the unthreaded portion 657. The clutch spring is configured to bias the externally threaded retractor 654 distally (e.g., toward the threads of the internally threaded tube 652) when the externally threaded retractor 654 has reached the end of the threads of the internally threaded tube 652.

In some implementations, continued rotation of the knob 626 following disengagement of the external threads of the externally threaded retractor 654 and the internal threads of the internally threaded tube 652 results in an audible indication that the device is in a closed position. The biasing of the externally threaded retractor 654 can also reduce slop in the threaded connection, thereby improving stability of the paddle angle. The biasing of the externally threaded retractor 654 towards the internal threads of the internally threaded tube 652 ensures that the externally threaded retractor 654 will be re-engaged by the internally threaded tube 652 when the knob 626 is rotated in an opposite direction that corresponds to advancement of the actuation element 112.

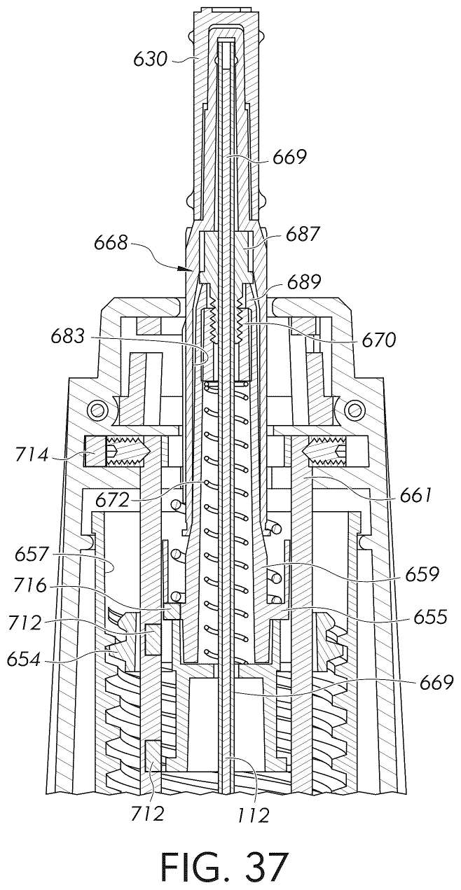

In some implementations, as shown in FIGS. 36 and 37, the externally threaded retractor 654 includes a central passage 683. A crimp assembly 668 is coupled in the passage 683. The crimp assembly 668 attaches the actuation element 112 that is in an actuation tube 669 to the paddle release knob 630. The crimp assembly 668 can take a variety of different forms. In the illustrated example, the crimp assembly 668 comprises a collet 687 and a nut 670. The collet 687 includes external threads and the nut includes internal threads. The collet and nut each have a central bore that is sized to receive the actuation tube 669 which surrounds the actuation element 112. The actuation element 112 can be fixed to the actuation tube 669 near the proximal end of the actuation tube 669. The actuation tube 669 is fixed to the crimp assembly 668, which is fixed to the externally threaded retractor 654. The external threads on the outer surface of the collet 687 engage with the internal threads of the nut 670. When tightened, the collet 687 and nut 670 fix the crimp assembly to both the actuation tube 669 and a shoulder 689 at the proximal end of the retractor 654. The nut is positioned within the central passage 683 of the externally threaded retractor 654. A spring 672 is also positioned within the central passage 683 of the externally threaded retractor 654 such that the nut 670 is biased against the shoulder 689. The positioning of the nut 670 against the shoulder 689 allows the collet 687 to be easily attached to the nut 670.

In some implementations, e.g., where control 626 is configured as a knob, the knob 626 can be rotated, which rotates the internally threaded tube 652 to rotate with respect to the housing 632, thereby driving the externally threaded retractor 654 axially. The axial movement of the externally threaded retractor 654 causes axial movement of the actuation element 112, which moves the implantable device between a fully elongated configuration, an open configuration, and a closed configuration. One or more sensors 712 can be disposed in the housing 632 to determine the configuration of the device (e.g., the fully elongated configuration, the open configuration, the closed configuration, positions therebetween, etc.). The sensors 712 can take a wide variety of different forms and can be configured and positioned in a wide variety of different ways to determine the configuration of the implantable device. The sensor(s) 712 can be hall effect sensors, proximity sensors, optical sensors, ultrasonic sensors, switches, magnetic sensors, etc.

In the example illustrated by FIG. 37, a plurality of sensors 712 are positioned along one of the guide rods 661 to enable the visual indicator 700 on the handle 616 to provide an indication regarding a location or configuration of the paddles of the implantable device. Each of the plurality of sensors 712 can be coupled to a microprocessor 714. The microprocessor 714 is further coupled to the visual indicator 700 (shown in FIG. 34). A sensed component 716, such as a magnet, material with distinct optical properties, material with distinct acoustic properties, or any other material or configuration that can be sensed by the sensor 712, is coupled to the externally threaded retractor 654. As the externally threaded retractor 654 is driven axially, the sensed component 716 also is driven axially, and stimulates one or more of the plurality of sensors 712 based on its axial position. The stimulated sensors 712 generate and output a signal that is received by the microprocessor 714. The microprocessor 714 generates and outputs a signal to the visual indicator 700 based on the signals received from the stimulated magnetic sensors 712, thereby causing the visual indicator 700 to provide a visual indication of the location of the paddles of the implantable device.

For example, in some implementations, when the implantable device is in a fully elongated configuration (e.g., the externally threaded retractor 654 is fully engaged with the internally threaded tube 652 and in a distal position), the sensed component 716 stimulates a first sensor 712 positioned at a distal end of the guide rod 661, which causes the visual indicator 700 to generate a first visual effect. The visual effect generated by the visual indicator 700 can vary depending on the particular visual indicator 700 employed. For example, the visual indicator 700 can be an LED indicator configured to illuminate in different colors. In some implementations, the visual indicator 700 can provide a green light to indicate that the implantable device is in a fully elongated configuration and is ready to be positioned. As the knob 626 is rotated and the externally threaded retractor 654 is driven in the proximal direction to move the implantable device from the fully elongated configuration to an open configuration for capturing leaflets, the sensed component 716 stimulates a second sensor 712 positioned on the guide rod 661.

In some implementations, the second sensor 712 is positioned proximally with respect to the first sensor. Stimulation of the second sensor 712 causes the visual indicator 700 to generate a second visual effect, such as a yellow light indicative of the implantable device being in an open configuration. The knob 626 is rotated further, thereby driving the externally threaded retractor 654 further proximally into a position in which it is at least partially disengaged from the internally threaded tube 652, as illustrated in FIG. 37. In this position, the sensed component 716 stimulates a third sensor 712 positioned at a proximal end of the guide rod 661, which causes the visual indicator 700 to generate a third visual effect. For example, the third visual effect can be a red light indicative of the implantable device being in a closed configuration.

In some implementations, the microprocessor 714 can receive signals from more than one sensor 712 at the same time and can, therefore, generate a signal based on more than one received signal. For example, the microprocessor 714 can receive signals from the first and second magnetic sensors when the magnet is positioned between the first and second magnetic sensors. In some implementations, the visual effect generated by the visual indicator can be an intensity of an illuminated light, such that when the implantable device is in a fully elongated configuration, the light is relatively dim and when the implantable device is in a closed configuration, the light is relatively bright.

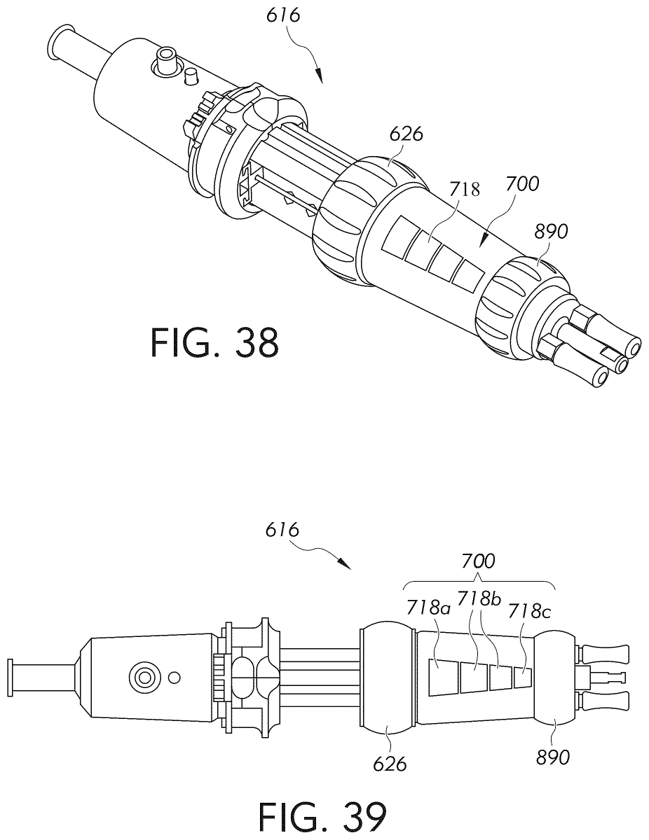

In some implementations, the visual indicator 700 can include a plurality of LEDs 718a, 718b, 718c (generally referenced by reference number 718) that are illuminated as a circuit is completed including the LED, as illustrated in FIGS. 38-40B. As shown in FIG. 38, the visual indicator 700 is positioned on the housing of the handle 616 between the control 626, which is configured to control the position of the actuation element 112 relative to the handle 616 and outer shaft 611 and a paddle width control 890, which is configured such that it can be actuated to move a width adjustment element to adjust the width of the paddles. Although in FIGS. 38 and 39 the visual indicator 700 includes four LEDs 718, in some implementations, other numbers of LEDs 718 can be included. In some implementations, three or more LEDs 718 are included: a distal LED 718a, one or more central LEDs 718b, and a proximal LED 718c. One or more of the LEDs can be illuminated as the control 626 is actuated (e.g., rotated, etc.) to indicate how open the paddles of the device are and/or as the control 890 is actuated (e.g., rotated, etc.) to indicate how wide/narrow paddles of the device are.

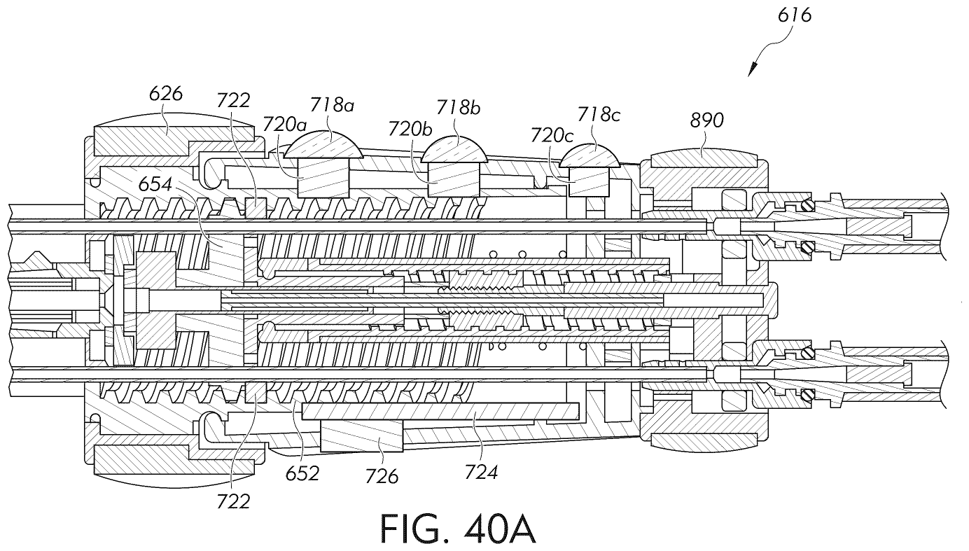

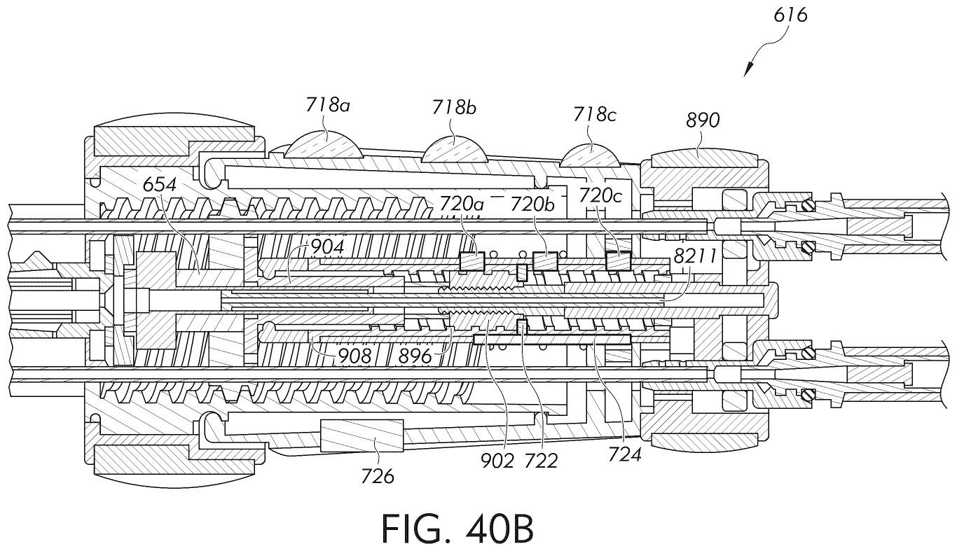

Turning now to FIG. 40A, an example including three LEDs, 718a, 718b, and 718c is shown. Each LED is coupled to a corresponding LED sensor, 720a, 720b, 720c. The sensors 720a, 720b, 720c can be contacts, hall sensors, magnetic sensors, proximity sensors, optical sensors, etc. A sensed component 722, such as a contact or conductive material, a magnet, material with distinct optical properties, material with distinct acoustic properties, or any other material, is coupled to the externally threaded retractor 654. In the example illustrated by FIG. 40A, the sensors 720a, 720b, 720c and the sensed component 722 are all contacts. Additionally, a circuit connecting strip contact 724 is positioned within the internally threaded tube 652. The sensed component 722 can be an annular ring that can contact both the connecting strip contact 724 and one or more of the sensors 720a, 720b, 720c (depending on the position of the sensed component 722). The circuit connecting strip contact 724 is electrically coupled to a battery 726. Each of the LED sensors 720a, 720b, 720c, the sensed component 722, and the circuit connecting strip contact 724 are formed from a metal or other conductive material. When one of the LED sensors 720a, 720b, 720c is in contact with the sensed component 722 and the circuit connecting strip contact 724, a circuit is created with the battery 726. The particular location of the LED sensors 720a, 720b, and 720c is selected to correspond to a particular configuration of the implantable device, or the location of the paddles. For example, LED sensor 720a can be positioned along the internally threaded tube 652 at a location that corresponds to a fully elongated configuration of the implantable device, LED sensor 720b can be positioned along the internally threaded tube 652 at a location that corresponds to an open configuration of the implantable device, and LED sensor 720c can be positioned along the internally threaded tube 652 at a location that corresponds to a closed configuration of the implantable device.

Accordingly, as the knob 626 is rotated and the externally threaded retractor 654 is driven with respect to the internally threaded tube 652, the sensed component 722 remains in contact with the circuit connecting strip contact 724. When the externally threaded retractor 654 is positioned adjacent to the LED sensor 720a, a circuit is completed, thereby causing the corresponding LED 718a to illuminate, indicating that the implantable device is in a fully elongated configuration. As the knob 626 is further rotated, driving the externally threaded retractor 654 proximally with respect to the internally threaded tube 652, the sensed component 722 moves out of contact with the LED sensor 720a, interrupting the circuit and causing the LED 718a to go out. Then, the sensed component 722 moves into contact with the LED sensor 720b, completing the circuit with the LED sensor 720b and causing the corresponding LED 718b to illuminate, indicating that the implantable device is in an open configuration. As the knob 626 is further rotated, driving the externally threaded retractor 654 proximally with respect to the internally threaded tube 652, the sensed component 722 moves out of contact with the LED sensor 720b, interrupting the circuit and causing the LED 718b to go out. Then, the sensed component 722 moves into contact with the LED sensor 720c, completing the circuit with the LED sensor 720c and causing the corresponding LED 718c to illuminate, indicating that the implantable device is in a closed configuration.

In some implementations in which the implantable device includes paddles having an adjustable width, visual indicators can be employed to indicate a width of the paddles in addition to, or as an alternative to, indicating the position of the paddles of the implantable device. In FIG. 40B, another implementation including three LEDs, 718a, 718b, and 718c is shown. As in FIG. 40A, each LED in FIG. 40B is coupled to a corresponding LED sensor 720a, 720b, 720c. However, in FIG. 40B, the sensed component 722 is coupled to a follower 902. The circuit connecting strip contact 724 is positioned within a guide 908. The circuit connecting strip contact 724 is electrically coupled to a battery 726. Each of the LED sensors 720a, 720b, 720c, the sensed component 722, and the circuit connecting strip contact 724 can be formed from a metal such that when one of the LED sensors 720a, 720b, 720c is in electrical communication with the sensed component 722 and the circuit connecting strip contact 724, a circuit is created with the battery 726. In the implementation shown, each of the LED sensors 720a, 720b, 720c is positioned on an inner surface of the guide 908 at a position opposite the circuit connecting strip contact 724. The particular location of the LED sensors 720a, 720b, and 720c is selected to correspond to a particular width of the paddles (e.g., fully widened, fully narrowed, or a median width of the paddles).

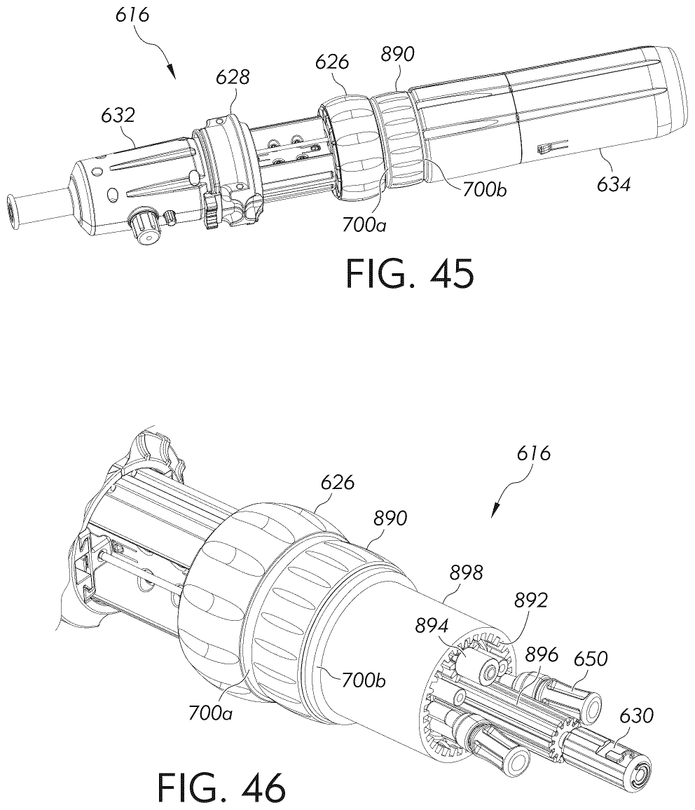

In some implementations, as is best shown in FIG. 46, the paddle width control 890 is configured as a rotatable knob that rotates about a central axis extending through the handle 616 without interfering with the clasp control tubes and/or clasp actuation lines 624 (shown in FIGS. 34 and 35) that extend to the suture locks 650 at the end of the handle 616. Depending on the implementation, the paddle width control knob 890 can be spaced apart from the control or knob 626, as shown in FIGS. 38-40B, or the paddle width control knob 890 can be positioned adjacent to the control/knob 626, as shown in FIGS. 45 and 46.

As best shown in FIGS. 46, in some implementations the paddle width control 890 is coupled to a planetary gearbox that includes a ring gear 892, a pair of planet gears 894 (one shown in FIG. 46), and an elongated central gear 896. Accordingly, actuation of the paddle width control 890 (e.g., rotation with respect to the handle 616, etc.) is effective to cause rotation of the elongated central gear 896, which in turn is effective to move the width adjustment element 8211 (FIG. 40B) in an axial direction, thereby adjusting the width of the paddles.

In some implementations, the ring gear 892 is housed within a barrel 898 that is fixed to the paddle width control 890 such that actuation or rotation of the paddle width control 890 turns the barrel 898 and the ring gear 892 fixed within the barrel 898. In the illustrated example, the axle of each planet gear 894 is fixed. Teeth of the ring gear 892 engage with the teeth of the pair of planet gears 894 such that rotation of the ring gear 892 causes rotation of the planet gear 894 on their fixed axles. The teeth of the pair of planet gears 894 also engage with teeth of the elongated central gear 896 such that rotation of the pair of planet gears 894 causes rotation of the elongated central gear 896.

In some implementations, as shown for example in FIG. 40B, the follower 902 includes external threading that is configured to engage with internal threads of the elongated central gear 896. However, the follower 902 is rotationally fixed such that as the elongated central gear 896 is rotated, the follower 902 is axially driven with respect to the elongated central gear 896. The follower 902 is further fixedly coupled to the width adjustment element 8211. Accordingly, axial movement of the follower 902 results in axial movement of the width adjustment element 8211, which is effective to adjust the width of the paddles, as described above.

In some implementations, the elongated central gear 896 is positioned within the handle 616 proximate the externally threaded retractor 654 and receives a shaft 904 extending from one end of the externally threaded retractor 654. The shaft 904 is rotatably coupled to the elongated central gear 896 adjacent the follower 902. The width adjustment element 8211 extends through a central passage of the shaft 904 and the externally threaded retractor 654. A clip (not shown) axially fixes the shaft 904 with respect to the elongated central gear 896. As described above, the externally threaded retractor 654 is rotationally fixed relative to the housing of the handle 616, and is configured to open and close the implantable device coupled to the handle 616 as it is driven axially. The elongated configuration allows the elongated central gear 896 to maintain meshing contact with the planet gears 894 as the elongated central gear moves axially with the externally threaded retractor 654. Rotation of the elongated central gear 896 is effective to drive the follower 902 axially with respect to the externally threaded retractor 654. The movement of the follower 902 relative to the externally threaded retractor 654 is effective to move the width adjustment element 8211 with respect to the actuation portion of the device, thereby adjusting the width of the paddles.

In some implementations, the guide 908 extends around the follower 902 and is configured to prevent or inhibit the follower 902 from rotating. The guide 908 is rotationally fixed with respect to the handle 616. The guide 908 and the follower 902 extend through a central passage of the elongated central gear 896. As described above, rotation of the elongated central gear 896 is effective to drive the follower 902 axially, since rotation of the follower 902 is prevented or inhibited by the guide 908. The external teeth of the elongated central gear 896 engage with the teeth of the pair of planet gears 894, one of which is shown in FIG. 46.

In some implementations, the planetary gearbox also includes a carrier (not shown). The carrier can be fixedly coupled to a portion of the handle 616, such as the housing, and provides support for the axels and gears (e.g., the pair of planet gears 894). The carrier and pair of planet gears can be positioned within the barrel 898. The ring gear 892 can also be positioned within the barrel 898 to engage with the pair of planet gears 894. The elongated central gear 896 extends between and engages with the pair of planet gears 894.

In some implementations, counter-clockwise rotation of the paddle width control knob 890 causes counter-clockwise rotation of the ring gear 892, which turns the pair of planet gears 894 counter-clockwise. The counterclockwise rotation of the pair of planet gears 894 rotates the elongated central gear 896 clockwise. As described above, the rotation of the elongated central gear 896 is effective to drive the width adjustment element 8211 axially, thereby adjusting the width of the paddles of the implantable device.

Accordingly, in some implementations, as the paddle width control knob 890 is rotated and the follower 902 is driven with respect to the guide 908, the sensed component 722 remains in contact with the circuit connecting strip contact 724. When the follower 902 is positioned adjacent to the LED sensor 720a, a circuit is completed, thereby causing the corresponding LED 718a to illuminate, indicating that the paddles are at a maximum width. As the paddle width control knob 890 is further rotated, driving the follower 902 proximally with respect to the guide 908, the sensed component 722 moves out of contact with the LED sensor 720a, interrupting the circuit and causing the LED 718a to go out. Then, the sensed component 722 moves into contact with the LED sensor 720b, completing the circuit with the LED sensor 720b and causing the corresponding LED 718b to illuminate, indicating that the paddles are at a median width. As the paddle width control knob 890 is further rotated, driving the follower 902 proximally with respect to the guide 908, the sensed component 722 moves out of contact with the LED sensor 720b, interrupting the circuit and causing the LED 718b to go out. Then, the sensed component 722 moves into contact with the LED sensor 720c, completing the circuit with the LED sensor 720c and causing the corresponding LED 718c to illuminate, indicating that the paddles are at a minimum width.

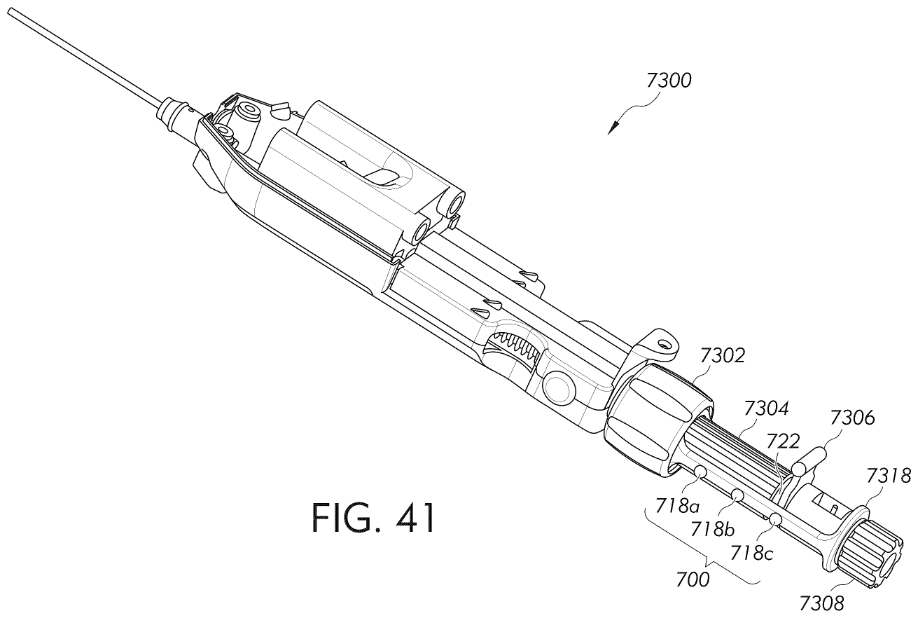

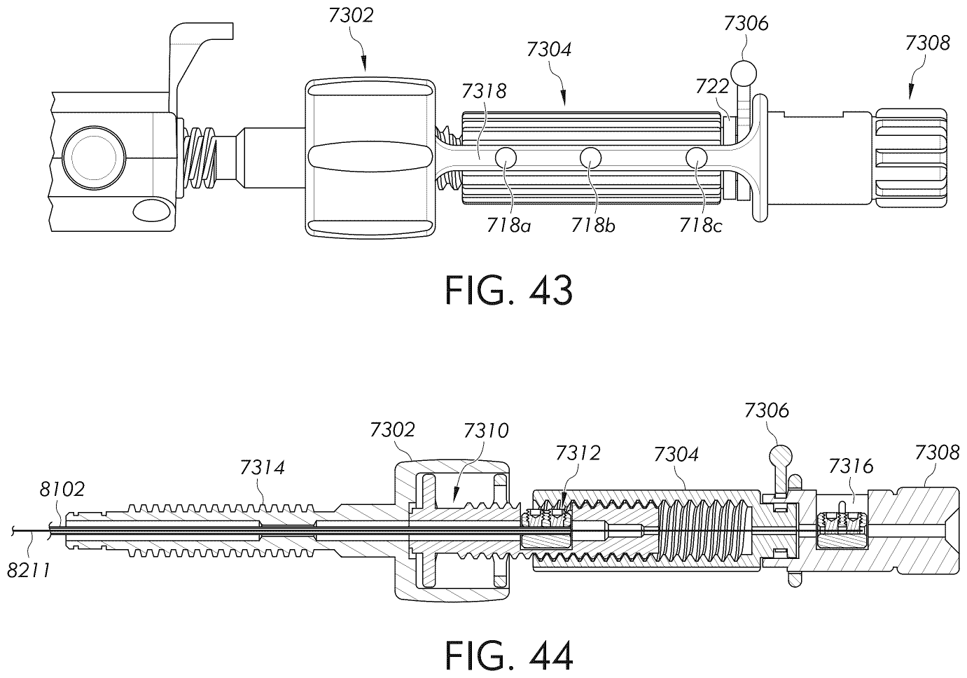

Turning now to FIGS. 41-44, an example including a visual indicator 700 in the form of a first LED 718a, a second LED 718b, and a third LED 718c is shown. The handle 7300 illustrated by FIGS. 41-44 can include a paddle actuation control 7302, a paddle width control 7304, a paddle release clip 7306, and a control element connection knob 7308. The handle 7300 can be an implementation of the handle 1616 of FIGS. 29-30, the paddle actuation control 7302 can be an implementation of the paddle actuation control 1626 in FIGS. 29-30, and the paddle width control 7304 can be an implementation of the paddle width control 1628 in FIGS. 29-30.

In some implementations, the paddle actuation control 7302 is rotatable about an axis extending longitudinally through the handle 7300. In some implementations, the paddle actuation control 7302 is configured as a rotatable knob, and the actuation element 8102 extends through the paddle actuation knob 7302 and is fixed to a ferrule 7312, as shown in FIG. 44. The ferrule 7312 is fixed to a frame 7310 that includes external threads for engaging with internal threads of the paddle width control knob 7304, as will be described in greater detail below. The distal end of the paddle actuation knob 7302 includes external threads 7314, which are configured to engage internal threads in a housing of the handle 7300. The paddle actuation knob 7302 is rotatable relative to both the housing of the handle 7300 and relative to the frame 7310 and attached ferrule 7312. Accordingly, as the paddle actuation knob 7302 is rotated about the axis with respect to the handle 7300, the paddle actuation knob 7302 axially drives the frame 7310 and attached ferrule 7312 (and, therefore, the actuation element 8102) with respect to the handle 7300, which is effective to axially drive the actuation element 8102 with respect to the device 8200.

In some implementations, the paddle width control knob 7304 is also rotatable about the axis extending longitudinally through the handle 7300. The width adjustment element 8211 extends through the actuation element 8102, the paddle actuation knob 7302, the ferrule 7312, and the paddle width control knob 7304. The width adjustment element 8211 is fixedly attached to a ferrule 7316 that is connected inside the control element connection knob 7308, as shown in FIG. 44. The ferrule 7316 fixes with width adjustment element 8211 rotationally and axially with respect to the control element connection knob 7308. The paddle width control knob 7304 is rotatably coupled to the control element connection knob 7308 and relative to the width adjustment element 8211.

In some implementations, as shown in FIG. 44, the paddle width control knob 7304 includes internal threads that engage with the external threads of the frame 7310. Accordingly, rotation of the paddle width control knob 7304 is effective to axially move the paddle width control knob 7304. Since the paddle width control knob 7304 is rotatably coupled to the control element connection knob 7308, the rotation of the paddle width control knob 7304 axially, but not rotatably, moves the width adjustment element 8211 with respect to the paddle actuation knob 7302. This is effective to axially drive the width adjustment element 8211 with respect to the attachment portion 8205 of the device 8200. Movement of the width adjustment element 8211 with respect to the attachment portion 8205 is effective to adjust the width of the paddles, as described hereinabove.

In some implementations, the frame 7310 includes a guide shaft 7318 that is positioned at least partially circumferentially around the paddle width control 7304 such that the paddle width control 7304 extends through the guide shaft 7318 along the longitudinal axis of the handle 7300. In FIGS. 41-43, the LEDs 718a, 718b, and 718c of the visual indicator 700 are positioned at different axial locations along the guide shaft 7318. As in the previous implementation, each of the LEDs 718a, 718b, and 718c can have a corresponding LED sensor (not shown) affixed to an inner surface of the guide shaft 7318. The LED sensors are positioned at locations corresponding to various widths of the paddles based on the particular implementation. For example, the first LED 718a can have a corresponding LED sensor at a location corresponding to a maximum paddle width, the second LED 718b can have a corresponding LED sensor at a location corresponding to a median paddle width, and the third LED 718c can have a corresponding LED sensor at a location corresponding to a minimum paddle width.

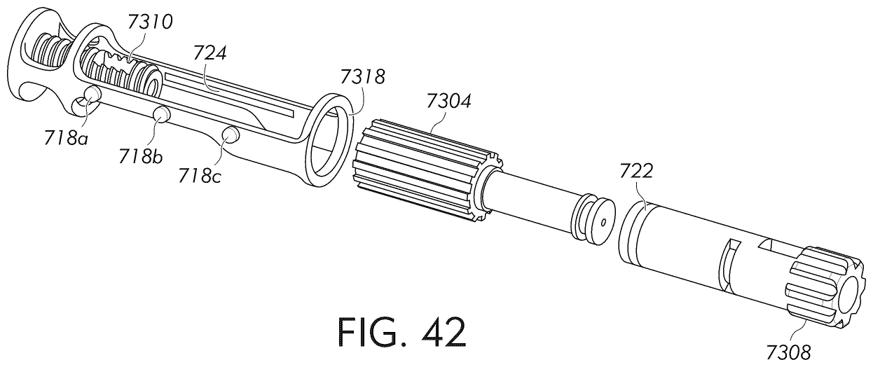

In some implementations, the guide shaft 7318 also includes a circuit connecting strip contact 724 on the inner surface of the guide shaft 7318, as shown in FIG. 42. The circuit connecting strip contact 724 is electrically coupled to a battery (not shown) to supply power to the circuit. A sensed component 722 is positioned on the paddle width control knob 7304. Accordingly, as the paddle width control knob 7304 is rotated and driven axially with respect to the frame 7310 and the guide shaft 7318, the sensed component 722 contacts one of the LED sensors while remaining in contact with the circuit connecting strip contact 724 to complete a circuit with the battery and cause the corresponding LED 718a, 718b, 718c to illuminate depending on the axial position of the paddle width control knob 7304.

Although the examples described in accordance with FIGS. 41-44 are described as incorporating individual LED sensors and a sensed component, it is contemplated that some implementations can utilize a magnetic sensor or an electronic slider switch to actuate the visual indicator based on the width of the paddles, as described hereinabove. For example, the LED contacts can be replaced with magnetic sensors, the sensed component can be replaced with a magnet, and a microprocessor can be incorporated to generate a signal effective to cause the LED to generate a visual effect based on the signals received from one or more of the magnetic sensors upon stimulation by the magnet.

In some implementations, a plurality of visual indicators can be incorporated into the handle. In FIGS. 45 and 46, handle 616 includes a first visual indicator 700a configured to provide a visual indication regarding a position of the paddles of an implantable device and a second visual indicator 700b configured to provide a visual indication regarding a width of the paddles of the implantable device. In the implementation shown in FIGS. 45 and 46, the paddle width control element is in the form of a paddle width control knob 890. The paddle width control knob 890 is configured as a rotatable knob that rotates about a central axis extending through the handle 616 without interfering with the clasp control tubes and/or clasp actuation lines 624 (shown in FIGS. 34 and 35) that extend to the suture locks 650 at the end of the handle 616 (covered by a cap 634 in FIG. 45). Each of the first and second visual indicators 700a, 700b is in the form of a ring adjacent to a corresponding knob (e.g., the knob 626 or the paddle width control knob 890) for which it is an indicator. However, it is contemplated that the visual indicators 700a, 700b can take other forms, such as one or more LEDs, an LED readout, or other displays. In some implementations, the visual indicator 700a can be in a first form while the visual indicator 700b can be in a second form that is different from the first form (e.g., one of the visual indicators is a plurality of LEDs configured to illuminate while the other of the visual indicators is an LED ring configured to change intensity). In some implementations, one or both of the visual indicators can be replaced with an auditory indicator, with the audible signals being different from one another to enable a user to distinguish one indication from another.

Some implementations have been described in which one or more visual indicators are incorporated into a handle of a delivery apparatus and configured to provide an indication regarding a configuration or position of the delivery apparatus or an implantable device coupled thereto. Although described as being configured as a visual indicator, the implementations described herein can be configured to include an auditory indicator as an alternative to a visual indicator. Moreover, although the implementations described above have set forth various combinations of actuation mechanism (e.g., electronic slider switches, magnetic sensors, or circuit contacts) with particular handle configurations and/or as an indicator of a particular configuration (e.g., a configuration of a catheter or sheath, a configuration of the implantable device, or a configuration of the paddles), it is contemplated that other combinations are contemplated and possible based on the disclosure provided herein.

The treatment techniques, methods, operations, steps, etc. described or suggested herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A steerable catheter assembly comprising:
a catheter with at least one control element that flexes the catheter;
a control handle coupled to the catheter, the control handle comprising:
a housing;
a control operatively coupled to the at least one control element wherein actuation of the control causes axial movement of the at least one control element with respect to the housing and moves the catheter between a first, straight configuration and a second, curved configuration; and
at least one visual indicator coupled to the at least one control element through the housing, and configured to provide an indication regarding a configuration of the catheter.

2. The steerable catheter assembly according to claim 1, wherein the control is configured as a control knob rotatable with respect to the housing of the control handle, and wherein rotation of the control knob causes axial movement of the at least one control element with respect to the housing and moves the catheter between the first, straight configuration and the second, curved configuration.

3. The steerable catheter assembly according to claim 1, further comprising:
an electronic slider switch electronically coupled to the at least one visual indicator, wherein actuation of the control axially moves the electronic slider switch thereby stimulating the at least one visual indicator.

4. The steerable catheter assembly according to claim 1, wherein the at least one visual indicator comprises a ring, one or more LEDs, an LED readout, or a visual display.

5. The steerable catheter assembly according to claim 1, wherein the indication comprises a variance of light intensity emitted by the at least one visual indicator, a variance of color of light emitted by the at least one visual indicator, or a variance of a number of LEDs illuminated.

6. The steerable catheter assembly according to claim 1, wherein the indication comprises an indication that the catheter is in the first, straight configuration.

7. The steerable catheter assembly according to claim 1, wherein the indication comprises an indication that the catheter is in the second, curved configuration.

8. A handle assembly for controlling an implantable device, the handle assembly comprising:
a handle housing;
a sheath extending distally from the handle housing;
an actuation element extending through the sheath, the actuation element coupleable to the implantable device;
a paddle actuation control operatively coupled to the actuation element, wherein actuation of the paddle actuation control causes axial movement of the actuation element with respect to the handle housing and the sheath, wherein the axial movement of the actuation element causes the implantable device to move between an open position and a closed position; and
at least one visual indicator coupled to the actuation element through the handle housing and configured to provide an indication regarding a configuration of the implantable device.

9. The handle assembly according to claim 8, wherein the paddle actuation control comprises a paddle actuation knob that is rotatable relative to the handle housing, wherein rotation of the paddle actuation knob causes the axial movement of the actuation element with respect to the handle housing and the sheath.

10. The handle assembly according to claim 8, wherein the at least one visual indicator comprises a ring comprising one or more LEDs positioned adjacent to the paddle actuation control.

11. The handle assembly according to claim 8, wherein the at least one visual indicator comprises one or more LEDs, an LED readout, or a visual display.

12. The handle assembly according to claim 8, wherein the indication comprises at least one of: a first indication that the implantable device is in the open position and a second indication that the implantable device is in the closed position.

13. The handle assembly according to claim 8, further comprising:
a microprocessor coupled to the at least one visual indicator,
at least one magnetic sensor positioned within the handle housing and coupled to the microprocessor, and
a magnet,
wherein the axial movement of the actuation element causes axial movement of the magnet with respect to the at least one magnetic sensor effective to stimulate the at least one magnetic sensor, and
wherein the microprocessor causes the at least one visual indicator to provide the indication responsive to stimulation of the at least one magnetic sensor.

14. The handle assembly according to claim 8, wherein the at least one visual indicator comprises a plurality of LEDs, each of the plurality of LEDs being coupled to a corresponding LED sensor, the handle assembly further comprising:
a circuit connecting strip contact,
a battery coupled to the circuit connecting strip contact, and
a sensed component,
wherein the axial movement of the actuation element causes axial movement of the sensed component with respect to the plurality of LED sensors to complete a circuit between the battery, the circuit connecting strip contact, the sensed component, and at least one of the plurality of LED sensors effective to cause the corresponding LED to illuminate, thereby providing the indication.

15. The handle assembly according to claim 8, wherein the indication comprises at least one of a variance of light intensity emitted by the at least one visual indicator, a variance of color of light emitted by the at least one visual indicator, and a variance of a number of LEDs illuminated.

16. A handle assembly for controlling an implantable device, the handle assembly comprising:
a handle housing;
a sheath extending distally from the handle housing;
an actuation element extending through the sheath, the actuation element configured to be coupled to the implantable device;
a paddle actuation control operatively coupled to the actuation element and configured such that actuation of the paddle actuation control causes axial movement of the actuation element with respect to the handle housing and the sheath, wherein the axial movement of the actuation element causes the implantable device to move between an open position and a closed position;
a paddle width adjustment element extending through the actuation element, the paddle width adjustment element configured to be coupled to at least one of a pair of paddles of the implantable device;
a paddle width control operatively coupled to the paddle width adjustment element and configured such that actuation of the paddle width control causes axial movement of the paddle width adjustment element with respect to the handle housing and the sheath, wherein the axial movement of the paddle width adjustment element causes a width of the at least one of the pair of paddles of the implantable device to be moved from a first width to a second width; and
at least one visual indicator coupled to the paddle width adjustment element, and configured to provide an indication regarding a configuration of the implantable device.

17. The handle assembly according to claim 16, wherein the paddle actuation control comprises a paddle actuation control knob that is rotatable relative to the handle housing, wherein rotation of the paddle actuation control knob causes axial movement of the actuation element with respect to the handle housing and the sheath.

18. The handle assembly according to claim 16, wherein the paddle width comprises a paddle width control knob that is rotatable relative to the handle housing, wherein rotation of the paddle width control knob causes axial movement of the paddle width adjustment element with respect to the handle housing and the sheath.

19. The handle assembly according to claim 16, wherein the at least one visual indicator comprises a ring, one or more LEDs, an LED readout, or a visual display.

20. The handle assembly according to claim 16, wherein the at least one visual indicator comprises a plurality of LEDs, each of the plurality of LEDs being coupled to a corresponding LED sensor,
the handle assembly further comprising:
a circuit connecting strip contact,
a battery coupled to the circuit connecting strip contact, and
a sensed component,
wherein the axial movement of the paddle width adjustment element causes axial movement of the sensed component with respect to the plurality of LED sensors to complete a circuit between the battery, the circuit connecting strip contact, the sensed component, and at least one of the plurality of LED sensors effective to cause the corresponding LED to illuminate, thereby providing the indication.
